(19) United States
(12) Patent Application Publication
(10) Pub. No.: US 2021/0128500 A1
(43) Pub. Date: May 6, 2021

(54) Methods of Treating Conditions Associated with Leaky Gut Barrier

(71) Applicant: The Regents of the University of California, Oakland, CA (US)

(72) Inventors: Pradipta Ghosh, San Diego, CA (US); Soumita Das, San Diego, CA (US)

(21) Appl. No.: 16/490,655

(22) PCT Filed: Mar. 5, 2018
(86) PCT No.: PCT/US2018/020910

§ 371 Date: Sep. 3, 2019

Related U.S. Application Data
(60) Provisional application No. 62/466,631, filed on Mar. 3, 2017.

(51) Int. Cl. A61K 31/17; A61P 1/00

(52) U.S. Cl. CPC A61K 31/17; C07K 16/24; A61P 1/00

(57) ABSTRACT

Methods to interrogate and modulate gut barrier integrity are provided. Methods for treating leaky gut barrier are also provided. Methods for early detection of diseases associated with inflammatory disorders. Methods to rapidly assess the effects of drugs, chemicals, nutritional supplements, vitamins, and probiotics on the integrity of the gut barrier are also provided.

### CROSS REFERENCE TO RELATED APPLICATION

This application claims the priority benefit of U.S. Provisional Patent Application No. 62/466,631, filed Mar. 3, 2017, the entire contents of which are incorporated herein by reference.

### GOVERNMENT SPONSORSHIP

This invention was made with government support under grants R01CA160911 and R01DK107585 awarded by the National Institutes of Health. The government has certain rights in the invention.

### FIELD OF THE INVENTION

This disclosure relates to methods for screening compounds such as drugs, nutritional supplements, and probiotics for their ability to enhance or disrupt the gut barrier. This disclosure also relates to methods for treating chronic illnesses associated with a leaky gut barrier.

### BACKGROUND

The gut is a complex environment; the gut mucosa maintains immune homeostasis under physiological circumstances by serving as a barrier that restricts access of trillions of microbes, diverse microbial products, food antigens and toxins to the largest immune system in the body. The gut barrier is comprised of a single layer of epithelial cells, bound by cell-cell junctions, and a layer of mucin that covers the epithelium. Loosening of the junctions induced either by exogenous or endogenous stressors, compromises the gut barrier and allows microbes and antigens to leak through and encounter the host immune system, thereby generating inflammation and systemic endotoxemia. An impaired gut barrier (e.g. a leaky gut) is a major contributor to the initiation and/or progression of various chronic diseases including, but not limited to, metabolic endotoxemia, type II diabetes, fatty liver disease, obesity, atherosclerosis, inflammatory bowel diseases, and cancers. Despite the growing acceptance of the importance of the gut barrier in diseases, knowledge of the underlying mechanism(s) that reinforce the barrier when faced with stressors is incomplete, and viable and practical strategies for pharmacologic modulation of the gut barrier remain unrealized.

In more detail, the intestinal barrier is the largest mucosal surface that separates diverse stressors (trillions of microbes, toxins, food antigens) on one side from the largest immune system on the other. The microbiomes in our gut [6-8] interact with the epithelium and affect the digestion and absorption of nutrients. Harmful microbes cause infections, systemic endotoxemia, and dictate our susceptibility to obesity, type II diabetes, and other chronic diseases [9-13]. Protective agents, such as commensal microorganisms (which can be mimicked by probiotics), as well as antimicrobial peptides and mucins that are synthesized by Paneth and goblet cells, respectively, are critical for maintaining the health of intestinal epithelial cells (IECs). The primary factor preventing free access of stressors to our immune cells is a single layer of IECs strung together in solidarity by cell-cell junctions. These junctions not only keep the toxic components out, but also allow absorption of drugs and essential nutrients. Thus, the precise orchestration of the gut barrier, i.e., IECs held together by tight junctions (TJs) most apically, adherens junctions (AJs) below these, and desmosomes below the AJs, is a fundamental necessity for gut development and barrier function and to withstand the constant bombardment by microbes/stressors. Evidence shows that dysfunction in the gut barrier can affect metabolism [12, 15], energy balance [12], gut permeability [16, 17], fatty liver disease [14], systemic endotoxemia and inflammation [15, 16, 18, 19], all components of obesity and metabolic syndrome [20-22]. In fact, the importance of the gut barrier in health and disease has gained so much traction in the past decade that it has ushered in the dawn of ‘Barriology’ [15] (defined by Shoichiro Tsukita as the science of barriers in multicellular organisms). Despite the growth in this area of research and discovery of plausible targets, e.g., MLCK [16], knowledge of the underlying mechanism(s) that reinforce the gut barrier during stress is incomplete, and pharmacologic modulation of the barrier is not currently a practical option in clinical practice.

In some aspects, there is a need for methods for treating chronic illnesses associated with a leaky gut barrier. In some aspects, there is a need for screening techniques to identify probiotics, drugs, or chemicals/nutrients having a beneficial effect of on the gut barrier.

### SUMMARY OF THE INVENTION

This disclosure provides methods for screening drugs, nutritional supplements, and probiotics for their ability to enhance or disrupt the gut barrier.

This disclosure also provides methods for treating chronic illnesses associated with a leaky gut barrier.

The present invention provides methods for treating a disease associated with leaky gut barrier in a patient comprising administering to the patient a pharmaceutical composition comprising a therapeutically effective amount of an AMP-activated kinase (AMPK) agonist.

In embodiments, the invention provides a method for treating a disease associated with leaky gut barrier in a patient comprising administering to the patient a pharmaceutical composition comprising a therapeutically effective amount of Metformin.

In embodiments, the invention provides a method for treating a disease associated with leaky gut barrier in a patient comprising administering to the patient a pharmaceutical composition comprising a therapeutically effective amount of a Metformin analogue.

In embodiments, the invention provides a method for treating a disease associated with leaky gut barrier in a patient comprising administering to the patient a pharmaceutical composition, wherein the pharmaceutical composition is a delayed release formulation of Metformin.

In embodiments, the invention provides methods for treating chronic endotoxemia in a patient comprising administering to the patient a pharmaceutical composition comprising a therapeutically effective amount of an AMP-activated kinase (AMPK) agonist.

In embodiments, the invention provides methods for treating a disease associated with leaky gut barrier in a patient comprising administering to the patient a pharmaceutical composition comprising a therapeutically effective amount of an AMP-activated kinase (AMPK) agonist, wherein the disease is selected from a group comprising; metabolic syndrome, obesity, type II diabetes, coronary artery disease, fatty liver, an inflammatory bowel disease, Crohn's disease, ulcerative colitis, allergy, food allergy, celiac sprue, childhood allergy, irritable bowel syndrome, Alzheimer's disease, Parkinson's disease, colorectal cancer, depression, and autism.

In embodiments, the invention provides methods for treating a disease in a patient, wherein the disease is associated with systemic infection and inflammation from having a leaky gut barrier, comprising administering to the patient a pharmaceutical composition comprising a therapeutically effective amount of an AMP-activated kinase (AMPK) agonist.

The present invention provides methods for identifying compounds with an ability to enhance or disrupt the gut barrier comprising, combining a candidate compound with an enteroid-derived monolayer, measuring or observing a signal associated with an AMPK→GIV stress-polarity pathway, and determining that the candidate compound activated the AMPK→GIV stress-polarity pathway.

In embodiments, the invention provides methods for identifying compounds with an ability to enhance or disrupt the gut barrier comprising, combining a candidate compound with an enteroid-derived monolayer, measuring or observing a signal associated with an AMPK→GIV stress-polarity pathway, and determining that the candidate compound activated the AMPK→GIV stress-polarity pathway, wherein the candidate compound is a synthetic or naturally occurring small molecule or protein, a nutritional supplement, a dietary component, a probiotic, a prebiotic, or a combination thereof.

In embodiments, the invention provides methods for identifying compounds with an ability to enhance or disrupt the gut barrier comprising, combining a candidate compound with an enteroid-derived monolayer, measuring or observing a signal associated with an AMPK→GIV stress-polarity pathway, and determining that the candidate compound activated the AMPK→GIV stress-polarity pathway, wherein the candidate compound is a synthetic or naturally occurring toxin or a substance of abuse selected from the group comprising nicotine, alcohol, and cannabis.

In embodiments, the invention provides methods for identifying compounds with an ability to enhance or disrupt the gut barrier comprising, combining a candidate compound with a human enteroid-derived monolayer, measuring or observing a signal associated with an AMPK→GIV stress-polarity pathway, and determining that the candidate compound activated the AMPK→GIV stress-polarity pathway.

In embodiments, the invention provides methods for identifying compounds with an ability to enhance or disrupt the gut barrier comprising, combining a candidate compound with an enteroid-derived monolayer comprising epithelial, goblet, Paneth, and enteroendocrine cells, measuring or observing a signal associated with an AMPK→GIV stress-polarity pathway, and determining that the candidate compound activated the AMPK→GIV stress-polarity pathway.

In embodiments, the invention provides methods for identifying compounds with an ability to enhance or disrupt the gut barrier comprising, combining a candidate compound with an enteroid-derived monolayer, measuring tight junction function or observing tight junctions associated with an AMPK→GIV stress-polarity pathway, and determining that the candidate compound activated the AMPK→GIV stress-polarity pathway.

The present invention provides methods for screening a compound for an ability to enhance or disrupt the expression of MCP-1 in gut epithelium comprising, combining a candidate compound with an enteroid-derived monolayer, measuring or observing a signal associated with an ELMO1→MCP-1 signaling axis, and determining whether the candidate compound activated the ELMO1→MCP-1 signaling axis.

In embodiments, the invention provides methods for screening a compound for an ability to enhance or disrupt the expression of MCP-1 in gut epithelium comprising, combining a candidate compound with an enteroid-derived monolayer, measuring or observing a signal associated with an ELMO1→MCP-1 signaling axis, and determining whether the candidate compound activated the ELMO1→MCP-1 signaling axis, wherein the candidate compound is a synthetic or naturally occurring small molecule or protein, a nutritional supplement, a dietary component, a probiotic, a prebiotic, or a combination thereof.

In embodiments, the invention provides methods for screening a compound for an ability to enhance or disrupt the expression of MCP-1 in gut epithelium comprising, combining a candidate compound with a human enteroid-derived monolayer, measuring or observing a signal associated with an ELMO1→MCP-1 signaling axis, and determining whether the candidate compound activated the ELMO1→MCP-1 signaling axis.

In embodiments, the invention provides methods for screening a compound for an ability to enhance or disrupt the expression of MCP-1 in gut epithelium comprising, combining a candidate compound with an enteroid-derived monolayer comprising epithelial, goblet, Paneth, and enteroendocrine cells, measuring or observing a signal associated with an ELMO1→MCP-1 signaling axis, and determining whether the candidate compound activated the ELMO1→MCP-1 signaling axis.

The present invention provides methods of identifying a compound with an ability to enhance or disrupt the expression of TNF-α in macrophages in the gut comprising, combining a candidate compound with an enteroid-derived monolayer, measuring or observing a signal associated with an ELMO1→TNF-α signaling axis, and determining that the candidate compound activated the ELMO1→TNF-α signaling axis.

In embodiments, the invention provides methods of identifying a compound with an ability to enhance or disrupt the expression of TNF-α in macrophages in the gut comprising, combining a candidate compound with an enteroid-derived monolayer, measuring or observing a signal associated with an ELMO1→TNF-α signaling axis, and determining that the candidate compound activated the ELMO1→TNF-α signaling axis, wherein the candidate compound is a synthetic or naturally occurring small molecule or protein, a nutritional supplement, a dietary component, a probiotic, a prebiotic, or a combination thereof.

In embodiments, the invention provides methods of identifying a compound with an ability to enhance or disrupt the expression of TNF-α in macrophages in the gut comprising, combining a candidate compound with a human enteroid-derived monolayer, measuring or observing a signal associated with an ELMO1→TNF-α signaling axis, and determining that the candidate compound activated the ELMO1→TNF-α signaling axis.

In embodiments, the invention provides methods of identifying a compound with an ability to enhance or disrupt the expression of TNF-α in macrophages in the gut comprising, combining a candidate compound with an enteroid-derived monolayer comprising epithelial, goblet, Paneth, and enteroendocrine cells, measuring or observing a signal associated with an ELMO1→TNF-α signaling axis, and determining that the candidate compound activated the ELMO1→TNF-α signaling axis.

The present invention provides methods of detecting a disease associated with inflammation due to luminal dysbiosis comprising, obtaining an epithelium sample from a subject and detecting ELMO1 levels in the epithelium sample from the subject, wherein increased ELMO1 levels in the subject compared to a healthy control indicate the presence of a disease associated with inflammation due to luminal dysbiosis.

The present invention provides methods of treating a disease associated with luminal dysbiosis in a patient comprising, administering to the patient a pharmaceutical composition comprising a therapeutically effective amount of a MCP-1 inhibiting agent.

In embodiments, the invention provides methods of treating a disease in a patient selected from an inflammatory bowel disease, Crohn's disease, and ulcerative colitis comprising, administering to the patient a pharmaceutical composition comprising a therapeutically effective amount of a MCP-1 inhibiting agent.

In embodiments, the invention provides methods of treating a disease associated with luminal dysbiosis in a patient comprising, administering to the patient a pharmaceutical composition comprising a therapeutically effective amount of an anti-MCP-1 antibody.

### BRIEF DESCRIPTION OF THE DRAWINGS

FIG. 1 is a schematic showing how tight junctions (TJs) of the intestinal epithelial cells maintain barrier integrity despite multiple stresses to prevent the entry of a variety of antigens via the paracellular pathway. Upper left: Electron micrograph shows the epithelial barrier components (TJ, tight junction; AJ, adherens junction; DB, desmosomes; Mv, microvilli). Leaky TJs have been associated with systemic endotoxemia, which predisposes to, or aggravates, a variety of diseases [5].

FIGS. 2A-2C show the AMPK-GIV Stress Polarity Pathway. FIG. 2A shows a schematic summarizing the role of GIV in the regulation of cell-cell junction stability during energetic stress. Exposure of epithelial cells to conditions that induce energetic stress result in depletion of cellular ATP stores and accumulation of AMP (step 1); the latter activates AMPK kinase (step 2). Once activated, AMPK phosphorylates GIV at S245 (step 3) triggering its localization to the cell-cell junction (TJs) via increased ability to bind TJ-associated microtubules [3] (step 4). Once localized to the cell-cell junctions, GIV has been shown [4] to bind AJ-localized protein complexes, e.g., α- and β-Catenins and E-cadherin and link the catenin-cadherin complexes to the actin cytoskeleton (steps 5 and 8). GIV has also been shown to bind TJ proteins, e.g., aPKC/Par3/Par6 complex [1] (step 6), and link these proteins to G proteins and the actin cytoskeleton [2](steps 7 and 8). FIG. 2B shows immunofluorescence assays that were carried out on polarized MDCK monolayers in the absence (left; normal) and presence of (right) energetic stress that was induced by glucose starvation. While occludin, a marker of TJs is seen in both conditions, GIV that is phosphorylated by AMPK at Ser(S)245 is seen exclusively after stress; phosphoGIV at TJs resists junctional collapse. FIG. 2C shows a schematic summarizing specific aims that can be investigated. The role of the stress polarity pathway in the maintenance of intestinal barrier integrity can be studied in Caco-2 monolayers and human enteroids using a combination of stressors (LPS, microbes, reactive O₂ species generated after exposure to H₂O₂, etc.) with or without various genetic, probiotic-induced and pharmacologic manipulations of the pathway components, namely, AMPK and GIV.

FIG. 3 shows that GIV^{-/-} mice show tell-tale signs of high AMPK signaling. GIV^{-/-} mice develop goblet cell hyperplasia by postnatal day #14 in large intestines (top; *) prior to developing vacuolar apical cysts (VACs) by postnatal day #21.

FIGS. 4A-4C show that Metformin protects TJs during E. coli infection. FIG. 4A shows enteroids isolated from the colon (left) in culture and enteroid-derived monolayers (EDMs, right). In FIG. 4B, TEER was measured across EDMs that were pre-treated or not with Metformin (1 μM, 18 h) prior to challenge with E. coli K12 strain for 8 h. Drop in TEER (Y axis) is plotted. A representative experiment is shown. Metformin pre-treatment significantly reduced the drop in TEER, indicating that TJs were preserved. FIG. 4C shows EDMs treated as in 4B, fixed, and stained for occludin (a TJ marker; green grey scales). Activation of the AMPK→GIV stress polarity axis was monitored using pS245GIV (red). TJs were better preserved (intact occludin pattern) in Metformin-treated samples. Arrowheads=separation of TJs and loss of pS245GIV.

FIG. 5 shows that Metformin protects TJs from stress-induced collapse. Mouse intestine-derived EDMs were exposed to indicated amounts of LPS (top) or H₂O₂ (bottom) after treating them or not with 1 μM Metformin. Fixed monolayers were assessed for TJs by staining for occludin (a TJ marker; green grey scales). Activation of the AMPK→GIV stress polarity axis was monitored using pS245GIV (red grey scales). In each case, TJs were better preserved (intact occludin pattern) exclusively in Metformin-treated samples. TEER measurements agree with IF findings (not shown).

FIGS. 6A-6B show a representative experiment screening for benefits of pre- or pro-biotics by specifically testing human milk oligosaccharide (HMO).

FIGS. 7A-7D show ELMO1 expressed in the gut epithelium, and its elevated expression in the gut correlates with inflammation. FIG. 7A shows the gene Expression Omnibus (GEO) repository queried for the patterns of expression of ELMO1 in publicly available cDNA microarrays (GDS1330/GSE 1710; performed using mucosal biopsy samples from sigmoid colons of normal healthy controls (n=11), patients with Crohn's disease (CD; n=10) and ulcerative colitis (UC; n=11). FIG. 7B shows the expression of ELMO1, MCP-1 and TNF-α determined by qRT-PCR on the RNA isolated from colonic biopsies obtained from healthy controls and patients with Crohn's disease or ulcerative colitis. FIG. 7C shows the association between the levels of ELMO1 and MCP-1 (CCL2) mRNA expression tested in a cohort of 214 normal colon samples. The gene expression data were obtained from multiple publicly available NCBI-GEO data-series and analyzed using Hegemon. Left: Graph displaying individual arrays according to the expression levels of CCL2 and ELMO1 in 214 normal colon tissues. Probe ID used for each gene is shown. Blue and red grey scales indicate samples stratified into high (n=127) vs low (n=87) ELMO1 groups using StepMiner algorithm. Middle: Box plot comparing the levels of ELMO1 between high vs low ELMO1 groups. Right: Box plot comparing the levels of MCP-1 between high vs low ELMO1 groups. FIG. 7D shows the expression of ELMO1 determined by IHC on biopsies obtained from healthy controls (normal colon; left) or patients with UC or CD (right). ELMO1-specific staining was seen in the normal gut epithelium and in the lamina propria. Compared to normal (left, lower) ELMO1 expression in the UC/CD-affected gut (right) is higher.

FIGS. 8A-8D show enteroid-derived monolayers as a model system to selectively interrogate the role of the gut epithelium in CD. FIG. 8A(i) shows enteroids isolated from colonic biopsies that were obtained from either healthy controls or patients with CD viewed by light microscopy. A representative image of spheroids (arrows) is displayed. FIG. 8A(ii) shows enteroid-derived monolayers (EDM) prepared from the enteroids via terminal differentiation viewed by light microscopy. A representative image of the EDM is shown. FIG. 8B shows the levels of expression of ELMO1 (75 kD) detected by immunoblotting of enteroids derived from the terminal ileum and sigmoid colon of a representative healthy subject; where α-Tubulin was used as a loading control. FIG. 8C shows the expression of ELMO1, MCP-1 and IL-8 measured in the EDMs isolated from colonic biopsies obtained from one healthy and three CD patients. Bar graphs display the fold change in expression normalized to the healthy control. FIG. 8D shows EDMs derived from colonic biopsies obtained from healthy subjects and from patients afflicted with CD were infected (right) or not (left) with AIEC-LF82 prior to fixation and stained for ZO-1 (red grey scales), a marker for TJs and nucleus (DAPI; blue). Disruptions in TJs is marked (arrowheads). In healthy EDMs, disrupted TJs are seen exclusively after infection with AIEC-LF82 (compare two upper images). In CD-derived EDMs, disrupted TJs were noted at baseline (lower left), almost to a similar extent as after infection with AIEC-LF82 (compare two lower images).

FIGS. 9A-9C show the engulfment (internalization) of AIEC-LF82 through epithelial TJs is impaired in ELMO1^{-/-} EDMs with reduced recruitment of lysosomal proteins to the sites of internalization. FIG. 9A shows the expression of ELMO1 protein assessed by immunoblotting in enteroids isolated from colons of WT and ELMO1^{-/-} mice. α-Tubulin was analyzed as a loading control. FIG. 9B shows WT and ELMO1^{-/-} EDMs infected with AIEC-LF82 for 3 h prior to assessment of bacterial internalization using gentamicin protection assay. Bar graphs display % internalization. Data represent the mean±S.D of three separate experiments. * indicates p≤0.05 as assayed by two-tailed Student's t test. FIG. 9C shows WT and ELMO1^{-/-} EDMs infected with AIEC-LF82 as in 9B, fixed, stained with ZO-1 (red grey scales), LAMP1 (green grey scales) and DAPI for nucleus, and analyzed by confocal imaging. Left: Maximum projection of Z-stacks of representative fields were shown. Insets in merged images represent magnified images and displayed at the bottom to zoom in at the point of bacterial entry through epithelial TJs. Lysosomes (marked by LAMP1) were aligned with the TJs (marked by ZO-1) in WT EDMs, but remain dispersed throughout the epithelial cell in ELMO1^{-/-} EDMs. Lysosomes were seen in close proximity to the invading bacteria exclusively in the WT EDMs. Right: RGB plots show distance in pixels between the internalized bacteria (blue grey scales) and the TJs of host cells (red grey scales) and lysosomes (green grey scales).

FIGS. 10A-10F show the induction of MCP-1 and recruitment of monocytes in response to AIEC-LF82 is blunted in ELMO1^{-/-} EDMs; compared to WT EDMs. FIG. 10A shows the levels of expression of MCP-1 measured by qRT-PCR in EDMs derived from WT and ELMO1^{-/-} mice after infection with AIEC-LF82 for 6 h. Bar graphs display fold difference in MCP-1; mean±S.D of three separate experiments. * indicates p≤0.05 as assayed by two-tailed Student's t test. FIG. 10B shows the infection-induced production of MCP-1 by WT and ELMO1^{-/-}. EDMs in 10A were measured by ELISA on the supernatant collected after 6 h post-infection. Data represent the mean±S.D of three separate experiments. FIGS. 10C-10D show the schematics of the EDM-monocyte co-culture model used to study monocyte recruitment. Either infected EDMs (WT or ELMO1^{-/-}) (FIG. 10C) or conditioned supernatant (FIG. 10D) collected from infected EDMs was placed in the lower compartment separated from monocytes (upper chamber) separated by porous inserts of TRANSWELL. The number of monocytes that migrated from the upper to the lower chamber by 12 h was counted. FIGS. 10E-10F show bar graphs displaying monocyte migration towards infected EDMs (FIG. 10E) or conditioned media (FIG. 10F) plotted as percent (%) normalized to that seen when using supernatant from WT EDMs. Data represent as mean±S.D of three separate experiments. * indicates p≤0.05 as assayed by two-tailed Student's t test.

FIGS. 11A-11D compare WT macrophages, ELMO1-deficient macrophages displaying an impairment in the engulfment of AIEC-LF82 and induction of TNF-α. FIG. 11A shows the internalization of AIEC-LF82 in control (Control shRNA) and ELMO1-depleted (ELMO1 shRNA) J774 cells assessed using gentamicin protection assay as in FIG. 9B. Bar graphs display % internalization observed at 3 h after infection. Findings are represented as mean±S.D of three separate experiments, normalized to Control shRNA. * indicates p≤0.05 as assayed by two-tailed Student's t test. FIG. 11B shows the intestinal macrophages isolated from wild type (WT) and ELMO1^{-/-} mice were infected with AIEC-LF82 for 1 h at 37° C. and internalization measured by the gentamicin protection assay. The average number of internalized bacteria (mean±S.D) was calculated and represented as % internalization. FIG. 11C shows the TNF-α produced by AIEC-LF82-infected J774 cells in FIG. 11A analyzed by ELISA with the ELISA after 3 h of infection. Data represent as mean±S.D of three separate experiments. * indicates p≤0.05 as assayed by two-tailed Student's t test. FIG. 11D shows the schematic summarizing the role of ELMO1 in coordinating inflammation first in non-phagocytic (epithelial) and subsequently in phagocytic (monocytes) cells of the gut. Epithelial ELMO1 is essential for the engulfment of invasive pathogens like AIEC-LF82 and for the induction of MCP-1 in response to such invasion. MCP-1 produced by the epithelium triggers the recruitment of monocytes, facilitating their recruitment to the site of infection. Once recruited, ELMO1 in monocytes is essential for the engulfment and clearance of invasive bacteria and for the production of pro-inflammatory cytokines such as TNF-α. MCP-1 and TNF-α released from the epithelial and monocytic cells initiates a chain reaction for the recruitment and subsequent activation of other monocytes and T-cells. The resultant storm of pro-inflammatory cytokines propagates diseases characterized by chronic inflammation. The role of ELMO1 in monocyte recruitment can be explored using the EDM-monocyte co-culture model shown in FIGS. 10C and 10D.

FIG. 12 shows the Boolean relationship between CLDN2 and AMPKα2 conserved in the colon.

FIG. 13 shows the proportion of patients with cancer for patients with and without IBD over time. The data shows a relatively higher occurrence of cancer in patients with IBD.

FIGS. 14A-14H show colorectal cancer's initiation and progression is associated with a ‘leaky’ gut barrier and inhibition of the Stress-Polarity Pathway (the AMPK-GIV axis). FIGS. 14A-14D show the Stress-Polarity Pathway, as determined by pS245-GIV assessed in adenomas and colon cancers by IHC. The pathway is active in early tubular and sessile serrated adenomas (top panels in FIGS. 14A and 14B) but is lost in advanced adenomas (FIGS. 14A-14C) and carcinomas (FIG. 14D). FIG. 14E depicts bar graphs displaying % lesions that are positive. FIG. 14F shows Boolean analyses of NCBI-GEO discovery RNA sequence dataset. The Boolean analyses identified an invariant fundamental link between tight junction leakiness and AMPK. The relationship between the mRNA expression levels of AMPKα1 and α2 and each tight junction protein was systematically analyzed in ˜1500 colon samples within the NCBI-GEO RNA sequence dataset applying the Hegemon software, where individual gene-expression arrays can be plotted on two-axis chart (FIG. 14F). FIGS. 14G-14H display box plots showing the differences between AMPKα2 (PRKAA2) and Claudin 2 (CLDN2) in each group. Findings were validated by IHC.

FIG. 15 shows the activation of the AMPK-GIV axis with Metformin prevents the increase in CLDN2 in response to IBD-associated microbes.

FIGS. 16A-16D show normal colon and adenomas have an inverse gene expression signature. FIG. 16A shows Boolean analyses of NCBI-GEO discovery RNA sequence dataset. FIG. 16B displays a box plot of the differences between AMPKα2 (PRKAA2) and Claudin 1 (CLDN1) in each group. FIGS. 16C-16D show that AMPKα2 and CLDN1 and CLDN 2 protein expression levels in cancers matches mRNA patterns.

FIG. 17 shows an exemplary adaption of the EDM model to a semi-high throughput format for determination of; (1) Trans-epithelial resistance (TEER); (2) permeability of FITC-dextran; (3) expression levels of markers by qRT-PCR of the polarized monolayer; (4) cytokines by ELISA from the basolateral supernatant; and (5) TJ proteins on the monolayers by staining and visualization by confocal microscopy.

FIG. 18 shows the fold activation using AMP (FIG. 18A) and AMPK agonist A769662 (FIG. 18B).

FIG. 19 shows the efficacy of AMPK agonist A769662 using a semi-high throughput method.

FIGS. 20A-20E show the effect of activation of AMPK using AMPK agonist A769662. FIGS. 20A-20B show that activation of AMPK preserves colon length in DSS-induced colitis. FIGS. 20C-20E show that activation of AMPK heals colonic mucosa in DSS-induced colitis.

### DETAILED DESCRIPTION

When introducing elements of the present invention or the preferred embodiment(s) thereof, the articles “a”, “an”, “the,” and “said” are intended to mean that there are one or more of the elements. The terms “comprising”, “including,” and “having” are intended to be inclusive and mean that there may be additional elements other than the listed elements.

Pharmaceutically active: The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug. As used herein, pharmaceutically active agents include synthetic or naturally occurring small molecule drugs and more complex biological molecules.

Pharmaceutically acceptable: The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

Pharmaceutically acceptable salt: The term “pharmaceutically acceptable salt” as used herein refers to acid addition salts or base addition salts of compounds, such as an AMPK agonist, in the present disclosure. A pharmaceutically acceptable salt is any salt which retains the activity of the parent compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Zurich, 2002; Berge et al., J Pharm. Sci. 66: 1, 1977). In some embodiments, a “pharmaceutically acceptable salt” is intended to mean a salt of a free acid or base of a compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

Pharmaceutically acceptable carrier: The terms “pharmaceutically acceptable carrier” as used herein refers to an excipient, diluent, preservative, solubilizer, emulsifier, adjuvant, and/or vehicle with which a compound, such as an AMPK agonist, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the sections below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as an infection or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Treating” or “treatment” or “alleviation” refers to therapeutic administration wherein the object is to improve the health of a patient, such as to slow down (lessen) if not cure the targeted pathologic condition or disorder, prevent recurrence of the condition, or prevent condition development. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient stabilizes or the disorder or condition stops worsening. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

The term “combination” refers to either a fixed combination in one dosage unit form, or a kit of parts for the combined administration where a compound and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, a subject in need refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

Other objects, advantages and features of the present invention will become apparent from the following specification taken in conjunction with the accompanying figures.

Studied in accordance with this disclosure are the importance of a novel molecular mechanism, the stress-polarity pathway, which fortifies epithelial tight junctions (TJs) against stress-induced collapse. Within this pathway, AMP-activated kinase (AMPK) phosphorylates GIV/Girdin, a multi-modular junctional scaffold, exclusively when epithelial monolayers are subjected to various stressors; this phosphoevent is necessary and sufficient for the barrier-protective functions of AMPK. An intact AMPK→GIV axis is essential for the barrier-protective roles of Metformin, the widely-prescribed metabolic disruptor and anti-diabetic drug. This pathway stabilizes TJs and protects the gut barrier from a variety of stressors. This is testable using monolayers of human colonic cells or monolayers of human gut-derived enteroids to determine the effects of gut microbes on the stress-polarity pathway. The disruptive effects of infectious pathogens and reactive oxygen species (ROS) and protective effects of AMPK agonists, probiotics and nutritional supplements, on the TJs of the gut are analyzable using a combination of cell and molecular biology, genetic manipulations, and physiological and morphological analyses. The broad objective is to recognize the mechanism(s) that enable the gut barrier to serve as the front line of a host-defense strategy. The insights obtained from this disclosure provide both an entirely new strategy to tackle chronic diseases by targeting the gut barrier and strategies for rapid screening of drugs and probiotics for their barrier-protective/destroying effects on the gut.

Embodiments in accordance with this disclosure dissect the importance of a specialized signaling mechanism initiated by the AMP-activated kinase (AMPK), called the stress-polarity pathway, which tightens the TJs and resists stress-induced collapse. While there was ample evidence that pharmacologic activation of AMPK by Metformin protects epithelial barriers from diverse stressors, the precise mechanism by which AMPK exerted this effect remained unclear until recently, when it was discovered that GIV(G-alpha interacting vesicle associated protein)/Girdin is as an essential downstream effector of AMPK at the TJs. Phosphorylation of GIV by AMPK at a single site is both necessary and sufficient to strengthen TJs and preserve cell polarity and epithelial barrier function in MDCK monolayers; this AMPK→GIV axis is also essential for the barrier-protecting action of Metformin. The importance of this pathway in the gut can be investigated and translated. Data using an enteroid model confirms that the AMPK→GIV stress-polarity pathway triggered by Metformin is operational in the gut epithelium and resists TJ-collapse when the epithelium is stressed with pathogens. The current thinking in the field of barriology is that chronic systemic endotoxemia, due to a compromised gut barrier in the setting of stress, impacts multiple diseases. This disclosure evidences that the AMPK→GIV stress-polarity pathway protects the barrier against stressors and provides that this can be leveraged using at least two different specific aims.

The Engulfment and cell motility protein 1 (ELMO1) is a microbial sensor that enables macrophages to engulf enteric bacteria, and coordinately mount inflammation while orchestrating bacterial clearance via the phagolysosomal pathway [100]. ELMO1 binds the Pattern Recognition Receptor (PRR) Brain Angiogenesis Inhibitor-1 (BAI1) which recognizes bacterial Lipopolysaccharide (LPS) [80]. BAI1→ELMO1 signaling axis activates Rac1 and induces pro-inflammatory cytokines Tumor necrosis factor-α (TNF-α) and Monocyte Chemoattractant protein 1 (MCP-1) [100, 103]. The BAI1—ELMO1 signaling axis regulates the expression of ATP-binding cassette transporter ABCA1 (member of the human transporter sub-family ABCA), also known as the Cholesterol Efflux Regulatory Protein (CERP) which is linked to the development of cardiovascular diseases (CVD) [104].

In the context of inflammatory bowel disease (IBD), BAI1-deficient mice has pronounced colitis and lower survival [105]. Genome wide association studies (GWAS) have revealed the association of single nucleotide polymorphisms (SNPs) in ELMO1 with IBD, rheumatoid arthritis (RA) kidney disease, and diabetic nephropathy [106-108]. ELMO1 is required for the induction of several pro-inflammatory cytokines (MCP-1, IL1-β, TNF-α) that are known to drive a plethora of inflammatory diseases including IBD, CVD and RA. Among them, MCP-1 is a chemokine, which plays a significant role in the recruitment of mononuclear cells to the site of inflammation; it is also one of the major cytokines involved in inflammatory diseases like IBD [109].

Although the role of ELMO1 in phagocytic cells is clear, its role in the non-phagocytic cells, i.e., the gut epithelium has not previously been resolved. This disclosure provides that the sensing of IBD-associated microbes by ELMO1 in the gut epithelium, the first line host defense that is breached by invading pathogens, can serve as an upstream trigger for immune cell-mediated cytokine storm. Stem cell based-enteroids was used as the model system to interrogate the role of ELMO1 in epithelial cells faced with the dysbiosis in Crohn's disease (CD) patients, and defined a specific need for the engulfment pathway in the induction of MCP-1. The generation of MCP-1 by the epithelium appears to be followed by monocyte recruitment at the site of inflammation. Subsequently, bacteria enter monocytes in an ELMO1-dependent manner and trigger the release of TNF-α, thereby, propagating the chronic inflammatory cascade that is the hallmark of IBD. The invention provides that targeting ELMO1 helps simultaneously blunt both the ELMO1-MCP-1 and the ELMO1-TNF-α signaling axes in the epithelium and the macrophages, respectively, to combat the inflammation in IBD.

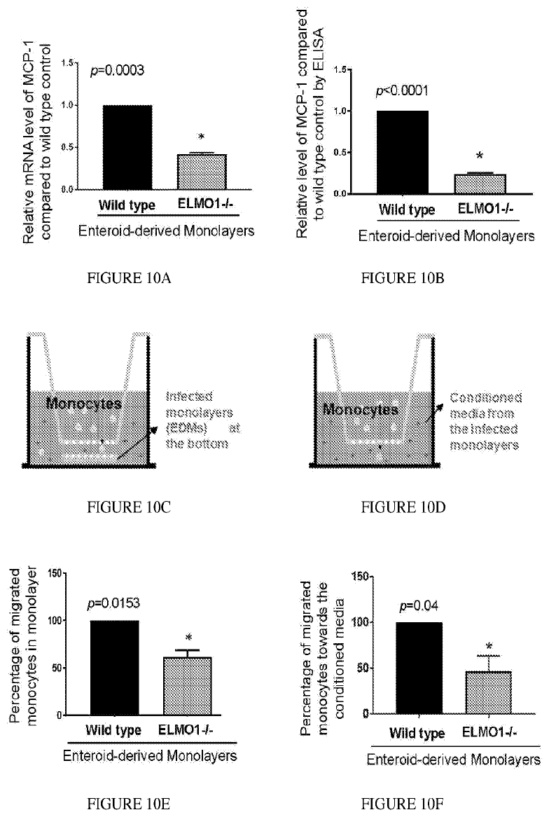

In embodiments, the disclosure provides a research tool to assess the 3-way interactions between microbes, gut epithelium, and the immune system. Using the EDM-monocyte co-culture model shown in FIGS. 10C and 10D a 3-step monocyte recruitment assay can be performed. The EDMs are adapted for co-culture in 2 chamber slides with IBD-associated microbes on the apical side and non-epithelial (immune and non-immune cells, e.g., monocytes, T-cells, myofibroblasts, etc.) on the basolateral side to recreate the 3-way system comprising microbes, gut epithelium, and the immune system. The impact of microbes on the epithelium, and the ability of the latter to release soluble factors on the basolateral side (cytokines such as MCP-1 or Butyrophillins, which attract γδ T-cells) can be assessed, alongside the measurement of how such factors trigger the recruitment and activation of non-epithelial cells. Using this approach the complex interplay between the gut microbes, the epithelium, and non-epithelial cells can individually be assessed for gene expression by RNA sequencing and cytokine expression by qPCR and ELISAs.

In embodiments, the 3-step monocyte recruitment assay uses enteroid-derived polarized monolayers (EDMs) from IBD or IBS patients or from polyps or colorectal cancers. In embodiments the EDMs is co-cultured with monocytes isolated from the same patient or conditioned supernatant from the infected EDMs incubated with the monocytes.

In embodiments, the disclosure provides a research tool to determine the effect of gut microbes on the stress-polarity pathway. To translate the findings on the stress-polarity pathway identified in MDCK monolayers to a relevant model, one can use the colonic epithelial cell line, Caco-2. TJ integrity in the setting of stress, with or without activation of the AMPK→GIV stress-polarity pathway can be assessed. Stressors can include pathogenic E. coli, bacterial LPS and reactive oxygen species generated by H₂O₂. The stress-polarity pathway can be modulated either directly using AMPK agonists (Metformin, AICAR, and A769662) or inhibitors (Compound C) or depletion of AMPK, or indirectly using probiotics like Akkermansia muciniphila. The latter is a mucin-degrading bacterium that increases in vivo during Metformin treatment [18] and has been shown to improve the gut barrier and render protection against a wide range of metabolic diseases [18-21]. Other nutrients (e.g., L-glutamine, butyrate, fatty acids) that are known to favor TJ integrity via AMPK [22-25] can also be tested. The specific role of GIV can be interrogated by depleting GIV by shRNA, and by stably expressing WT, phosphomimic or non-phosphorylatable GIV constructs. TJ integrity is monitored by measuring transepithelial electrical resistance (TEER), permeation of fluorescent dextran, confocal imaging of junctional markers (occludin and ZO-1), and electron microscopy. Activation of the stress-polarity pathway can be assessed by monitoring active phospho-AMPK and GIV by confocal IF.

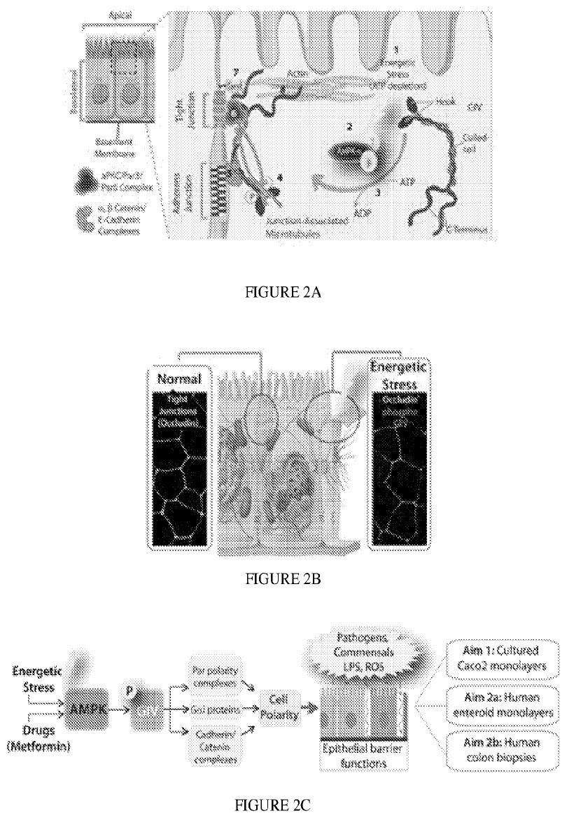

In embodiments, the disclosure provides a research tool to define the role of the stress polarity pathway in the human gut. To translate findings into a more physiologic system, one can carry out similar experiments as described in FIG. 2C, except, using human enteroid-derived monolayers (EDMs). One can also analyze colon biopsies from patients with or without chronic Metformin treatment by immunohistochemistry (phospho-AMPK, phospho-GIV, occludin, ZO-1). The biopsy-derived EDMs can be generated and assessed in vitro for TJ integrity. Because EDMs are an ideal model for studying epithelial physiology because four different cell types (epithelial, goblet, Paneth cells, and enteroendocrine cells) are present, thereby mimicking the native intestine, the relevance of these findings can be directly translated and adapted for screening drugs, compounds, and probiotics.

In embodiments, the disclosure provides a research tool to define the role of the engulfment pathway in the epithelium in the human gut. To translate findings into a more physiologic system, one can carry out the experiments using human enteroid-derived monolayers (EDMs). One can also analyze colon biopsies from patients. The biopsy-derived EDMs can be generated and assessed in vitro for TJ integrity. Because EDMs are an ideal model for studying epithelial physiology because four different cell types (epithelial, goblet, Paneth cells, and enteroendocrine cells) are present, thereby mimicking the native intestine, the relevance of these findings can be directly translated and adapted for screening drugs, compounds, and probiotics.

In embodiments, the disclosure provides a method for early detection of activation of the engulfment pathway associated with early inflammation due to luminal dysbiosis by detecting the levels of ELMO1 in the epithelium.

The present disclosure, among other things, provides a new molecular pathway in the gut barrier, a new strategy to treat chronic diseases, an important indication for Metformin, and allows for rapid screening of other drugs and probiotics for their barrier-protective/destroying effects in the gut.

### Examples

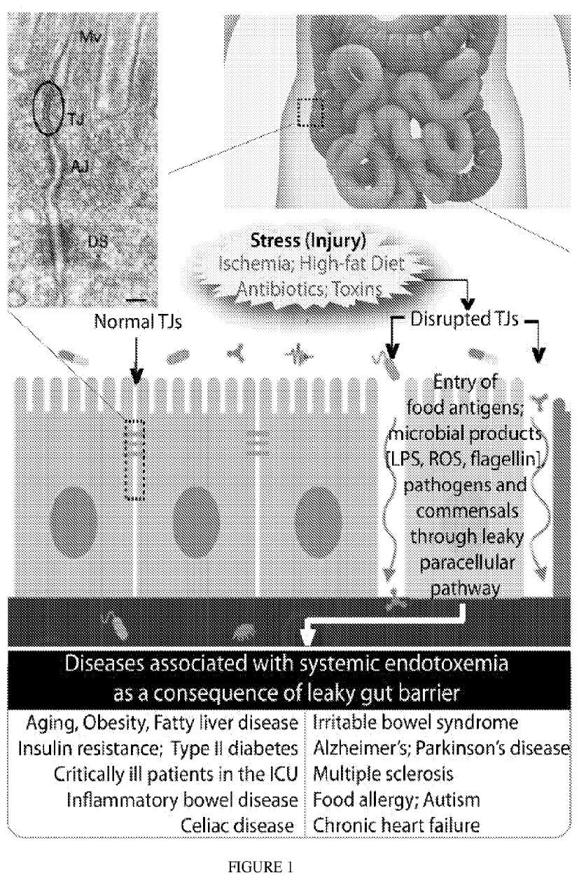

The Gut Lining. The tight-junctions (TJs) of an intact gut barrier protect people against potential barrier disruptors, e.g., hypoperfusion of the gut, microorganisms and toxins, over-dosed nutrients (high fat), drugs, and other elements of lifestyle (FIG. 1). On the other hand, this barrier must permit the absorption of essential fluids and nutrients. Antimicrobial products and mucins synthesized by Paneth and goblet cells, respectively, also serve as protective components of the gut barrier. A compromised gut barrier allows microbes and unwanted antigens to cross the epithelium and generates inflammation (systemic endotoxemia), which may contribute to a variety of diseases [5, 26-40] (listed in FIG. 1).

The Stress Polarity Pathway: reinforcement of epithelial TJs when under attack. Maintenance of apicobasal polarity in the gut epithelium requires the coordination of multiple sets of unique signaling pathways, whose integration in space and time dictates overall epithelial morphogenesis [41]. Among the evolutionarily conserved pathways that control epithelial cell polarity, several collaborate to assemble, stabilize and turn over the cell-cell junctions, e.g. CDCl42 and PAR proteins, such as the PAR3-PAR6-aPKC complex [42], and pathways that regulate membrane exocytosis and lipid modifications [42, 43]. In 2006-2007 three studies [3, 44, 45] simultaneously reported the existence of a special pathway for maintaining epithelial polarity; however, this pathway is triggered exclusively in the face of environmental stressors. In this pathway, AMPK, a key sensor of metabolic stress, stabilizes TJs, preserves cell polarity, and thereby maintains epithelial barrier function. Subsequent evidence has shown that pharmacologic activation of AMPK by Metformin protects the epithelial barrier against multiple environmental and pathological stressors. However, the mechanism by which AMPK protects the epithelium remained unknown until recently when GIV(G-alpha interacting vesicle associated protein)/Girdin was identified as a novel effector of AMPK at cell-cell junctions. By demonstrating that GIV is a direct target and an effector of the energy sensing kinase AMPK, the stress polarity pathway is defined at a greater resolution. It was shown that energetic stress triggers localized activation of AMPK at tricellular TJs, which mark the most vulnerable cell-cell contacts in sheets of polarized cells. Activation of AMPK triggers phosphorylation at a single site within GIV, i.e., Ser(S)245. Once phosphorylated by AMPK, pS245-GIV preferentially localizes to bicellular and tricellular TJs. Such localization is seen exclusively during TJ turnover, i.e., localization is seen both during TJ assembly as cells come in contact to form a monolayer and during TJ-disassembly as monolayers collapse in response to energetic stress or Ca²-depletion. These findings led to the conclusion that phosphorylation on GIV S245 is a key determinant of normal epithelial morphogenesis-phosphorylation favors polarized normal cysts, whereas absence of phosphorylation favors branching tubules and multi-lumen structures that are associated with loss of cell polarity. Finally, it was shown that pS245-GIV, which is generated only when the AMPK-GIV axis is intact, is both necessary and sufficient to fortify TJs, avoid junctional collapse and preserve cell polarity in the face of energetic stress. It was further concluded that a significant part of the junction-stabilizing effects of the AMPK agonists, AICAR and Metformin, during energetic stress [44, 45] is mediated by AMPK via its downstream effector, pS245-GIV. In demonstrating these findings, an unanticipated link between stress-sensing components and cell polarity pathways was revealed, and light was shed on how epithelial monolayers are protected despite being constantly bombarded by energetic stressors (see legend of FIGS. 2A-2B for mechanism of action of GIV at TJs). Briefly, phosphorylation of GIV at S245 localizes GIV to TJ-associated microtubules by enabling it to bind the ‘free’ C-term of α-Tubulin. Once localized, the polarity-scaffold GIV binds and activates the Par3/Par6/aPKC polarity complex and trimeric Gi proteins, and regulates catenin-cadherin complexes.

Pathophysiologic implications of the AMPK-GIV stress signaling pathway in the gut. Over the years, the beneficial (protective) effects of multiple nutritional components and dietary supplements (L-glutamine, butyrate, poly-unsaturated fatty acids; PUFA), and pharmacologic agents such as the widely-prescribed AMPK-activator, Metformin, on intestinal permeability in health and disease has been investigated; all studies converge on AMPK activation as a common pre-requisite for strengthening the gut barrier and reducing permeability (summarized in [5]). These studies raised the possibility that the AMPK-GIV stress polarity pathway [17] may affect a variety of diseases that are associated with increased intestinal permeability (FIG. 1). All these diseases are characterized by systemic inflammation due to chronic endotoxemia, presumably due to the translocation of endotoxins from the gut lumen into the circulation.

Among the diseases where the contribution of systemic endotoxemia has gained traction, evidence of causality in metabolic diseases, obesity, and type II Diabetes stands out prominently. Accumulating evidence shows that gut barrier dysfunction can influence whole-body metabolism [13, 46] by affecting energy balance [13], permeability [47, 48], metabolic endotoxemia [49] and inflammation [46, 47, 49, 50]; all of these contribute to the spectrum of disorders associated with metabolic syndrome [51-53]. Numerous studies using the AMPK-activator, Metformin, squarely implicate the AMPK-dependent stress polarity pathway as a major therapeutic target in these metabolic disorders [19, 54, 55]. Metformin enhances gut barrier integrity, attenuates endotoxemia and enhances insulin signaling in high-fat fed mice which likely contributes to the beneficial effects of Metformin on glucose metabolism, an enhanced metabolic insulin response, and reduced oxidative stress in the liver and muscle of mice [54]. Clinical trials using a delayed release formulation of Metformin (Metformin DR, which is designed to target the lower bowel and limit systemic absorption) have shown that Metformin works largely in the colon; despite the reduced absorption of Metformin DR, this formulation was effective in lowering blood glucose [55]. Metformin treatment directly impacts the colonic mucosa and the gut microbiome [18]; the number of goblet cells and mucin production increases, senescence is reduced, and Akkermansia muciniphila, a mucin-degrading bacterium that resides in the mucus layer, becomes abundant. The presence of this bacterium directly correlates with gut barrier integrity [19, 21] and inversely correlates with body weight and visceral adiposity in rodents and humans [19]. These studies have challenged conventional thinking regarding metabolic disease, emphasizing the importance of the gut barrier as the primary defect [56-58]. These studies also highlighted the effectiveness of Metformin as a potential therapeutic strategy to reinforce the gut barrier and correct metabolic disorders.

In embodiments, the invention provides a therapeutic strategy and the basis of a screening platform for drugs that tighten the gut barrier and reverse the metabolic syndrome. The invention answers a fundamental question, i.e., whether the AMPK→GIV stress polarity pathway plays a role in maintaining the integrity of the gut barrier that is constantly faced with metabolic/environmental stress, commensal and pathogenic microbes (FIG. 2C). It is understood that pharmacologic activation of AMPK by Metformin, AICAR, or by probiotics (like A. muciniphila) resists the collapse of epithelial TJs after challenge with injuries (LPS, pathogens, ROS) in monolayers of cultured cells (FIG. 2C), or enteroid monolayers grown on MATRIGEL (FIG. 2C). The AMPK→GIV axis is active in the native human colon and its activation amongst patients taking Metformin correlates with better glycemic control, and the presence or absence of fatty liver disease and/or obesity (FIG. 2C). The disclosure provides that the AMPK→GIV pathway represents a legitimate molecular mechanism by which TJs of IECs resist collapse when faced with stressors (i.e., gut microbiota) that trigger chronic metabolic diseases.

Valuable Insights from GIV^{-/-} Mice: The first clues that GIV affects the gut came from the published phenotypes of GIV^{-/-} mice [59-61]. Born grossly normal, they fail to gain weight, feed poorly, and die ˜3-4 weeks after birth. The large intestines of GIV^{-/-} mice are indistinguishable from WT littermates at birth, but begin to show polarity-defects by 2-3 wks., as determined by EM morphology (FIG. 3).

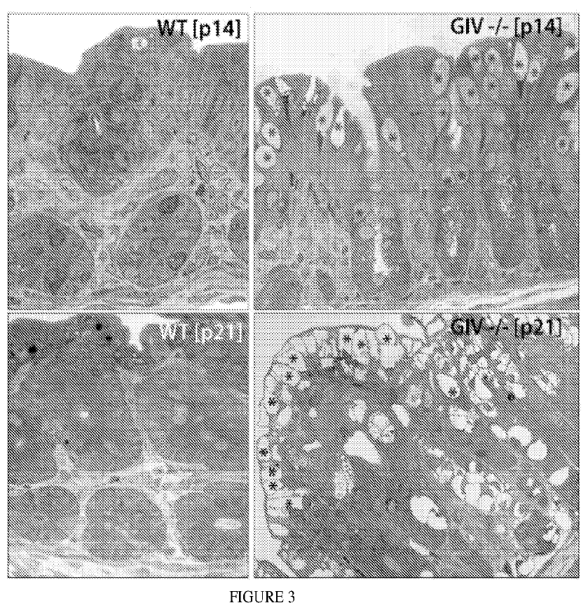

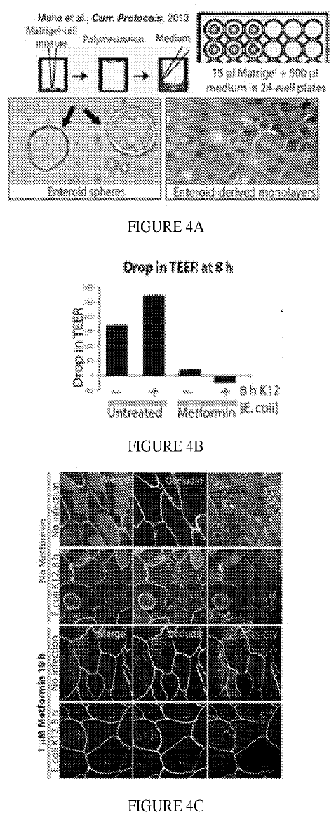

Murine and human enteroids as model systems to study gut barrier dysfunction: A therapeutic strategy to combat systemic endotoxemia by targeting the leaky gut barrier can impact a variety of diseases (FIG. 1). Prior to this disclosure's work, such a strategy remained unrealized, partly due to the unavailability of robust model systems that could be utilized for screening purposes. Sato et al. first developed defined conditions for the growth and expansion of intestinal stem cells as 3D intestinal organoids or enteroids [65]. Enteroids mimic the in vivo situation with four different cell types epithelial, goblet, Paneth cells, and enterocytes. The 3D-spheres (FIG. 4A; left) with 200-400 epithelial cells can be dissociated and plated onto TRANSWELLs as monolayers known as enteroid-derived monolayers (EDM) (FIG. 4A; right). EDMs maintain a similar architecture to enteroids with the same percentages of cells that exist in vivo [66, 67]. Furthermore, polarized cells in EDMs allowed access to the apical and basolateral sides separately [66, 67]. Thus, EDMs are an ideal model system for understanding the function of the stress polarity pathway in the presence of stress/infection.

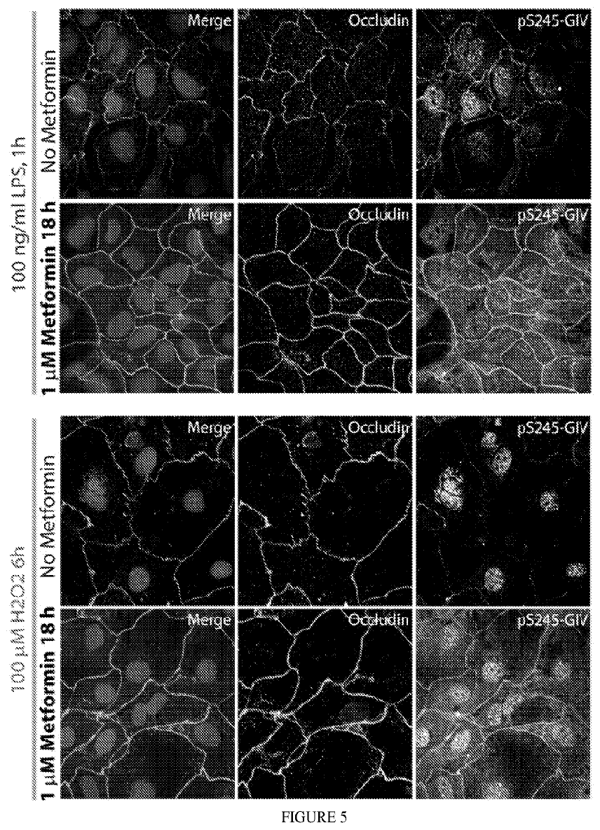

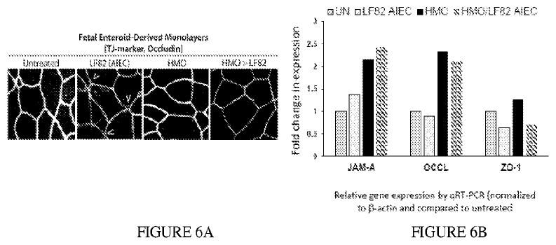

The developed model system can be used for screening purposes, and in an exemplary embodiment the benefits of human milk oligosaccharides treatment were screened (FIG. 6) and exemplary gene expression levels determined.

Metformin reinforces the gut barrier in the face of microbial infections: This disclosure successfully developed the enteroid and EDM system (FIG. 4A) from both human biopsies and from mouse intestine. Results using mouse and human EDMs showed that: 1) the AMPK→pS245GIV axis is active in EDMs; 2) pretreatment of EDMs with Metformin protects TJs against stress-induced collapse after treatment with E. coli (FIGS. 4B-C), LPS (FIG. 5; top), or H₂O₂ (FIG. 5; bottom), as determined by occludin staining, and that such protection was invariably associated with the increased presence of pS245GIV at the TJs.

Metformin reactivates the AMPK→pS245GIV axis: In non-diseased colons the stress-polarity pathway displays varying degrees of activity, with activity generally decreasing with age. Results using human EDMs showed that: 1) the activity of the AMPK→pS245GIV axis can be increased with Metformin in non-diseased aged colons; 2) reactivation of the AMPK→pS245GIV axis protects against invasive microbes associated with IBD in non-diseased aged colons.

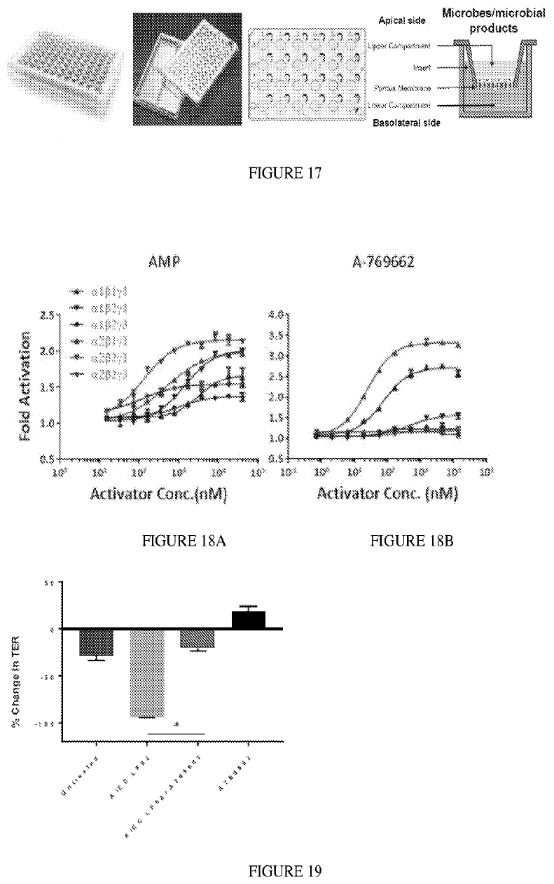

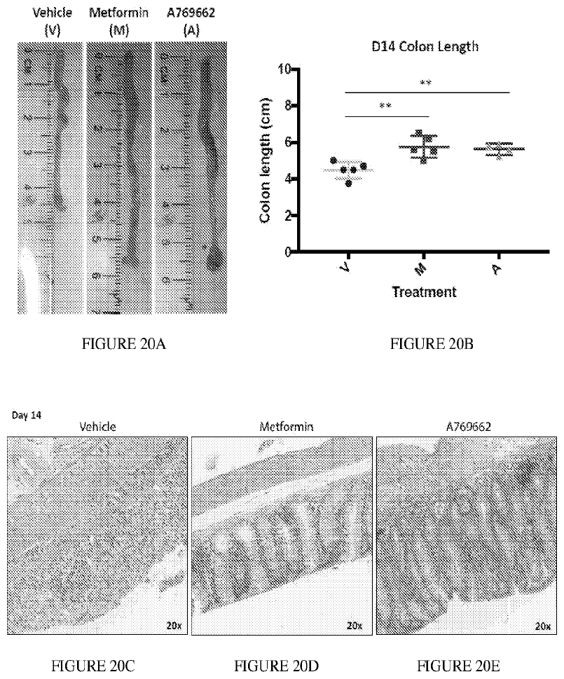

AMPK agonist: A769662 reactivates the AMPK→pS245GIV axis: The estimated AC₅₀ values of A769662 for α1β1γ1 and α2β1γ1 were 72.24 and 24.68 nM respectively, whereas the AC₅₀ values for 32-subunit-containing isoforms were >40 μM. (FIG. 18). The efficacy (FIG. 19) of A769662 was screened using the disclosed semi-high throughput method (FIG. 17). Data showed that activation of AMPK with A769662 preserves colon length and heals colonic mucosa in DSS-induces colitis (FIGS. 20A and 20 B).

Activation of the AMPK→pS245GIV axis suppresses dysplasia cancer progression: Claudin-2 (CLDN2) is consistently upregulated across all diseases associated with a gut barrier defect, such as but not limited to, Crohn's disease, ulcerative colitis, Celiac disease, and HIV.

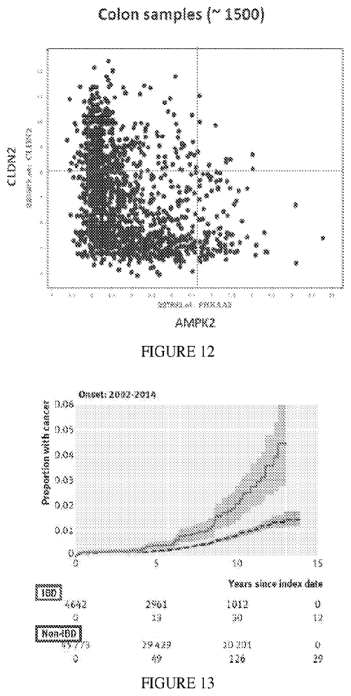

To investigate the relationship of CLDN2 in the stress-polarity pathway, the relative expression levels of CLDN2 and AMPKα1 and α2, respectively, was explored using the Hegemon software to generate scatter plots of 45,000 Affymetrix human microarrays downloaded from NCBI's Gene Expression Omnibus and normalized together. Among all the tight junction proteins the generated scatter plots revealed a Boolean relationship between CLDN2 and AMPKα2 that was not also present between CLDN2 and AMPKα1. This relationship between CLDN2 and AMPKα2 is conserved in the colon. (FIG. 12).

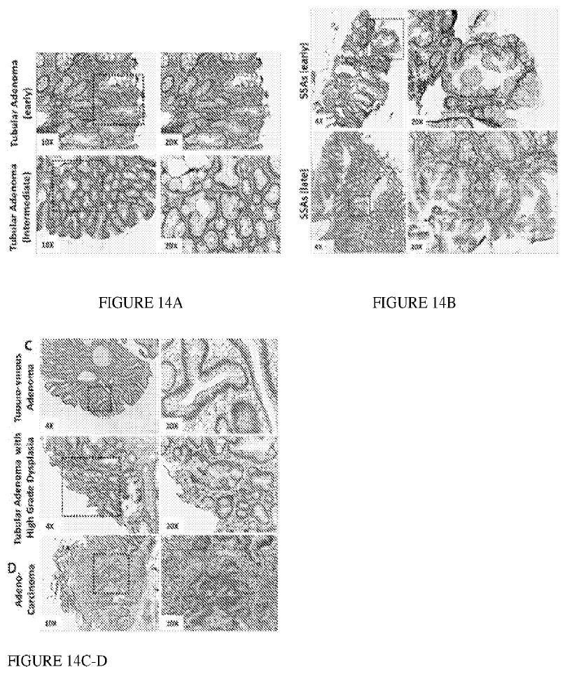

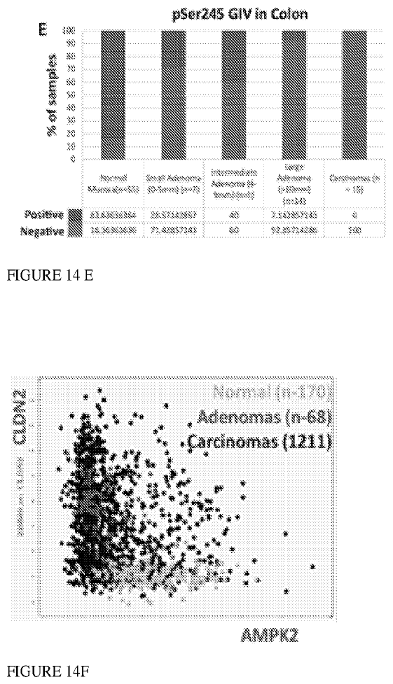

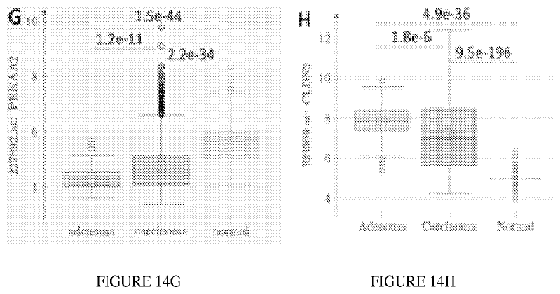

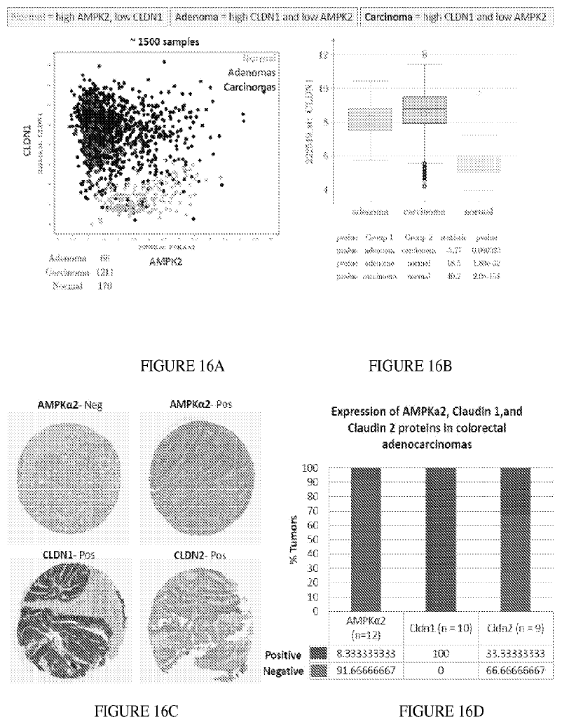

Data show that the proportion of patients with dysplasia cancer is higher in patients suffering from IBD (FIG. 13). Results using human EDMs showed that the activity of the AMPK→pS245GIV axis is progressively silenced during colorectal cancer and advanced sessile serrated polyps (FIG. 14). Boolean relationships indicate that AMPKα2 and CLDN2 expression are mutually exclusive in the colon. Moreover, findings demonstrate that normal-to-adenomatous transformation in the colon is associated with a reduction in AMPKα2 and a concomitant increase in the epithelial TJ protein, CLDN2. The latter is the only TJ protein that is invariably upregulated in IBD and other causes of leaky gut. No such association was seen with AMPKα1. However, a similar relationship between CLDN1 and AMPKα2 is seen in cancers (FIG. 16).

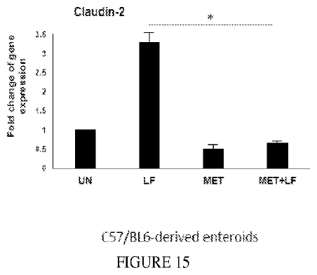

Activation of the AMPK→pS245GIV axis with Metformin prevents the increase in the CLDN2 biomarker in response to IBD associated microbes (FIG. 15).

This disclosure is innovative both conceptually and technically. First, the concept of modulating a molecular pathway (i.e., the stress-polarity pathway nucleated by AMPK) to increase or decrease the gut barrier function in response to microbes/other stressors is novel; there is no viable and practical strategy to do that yet. Such strategies impact diverse diseases that are fueled by a leaky gut. Second, this disclosure uses a novel model (enteroid-derived monolayers) to study the therapeutic potential of the stress-polarity pathway. The model can be adapted to screen nutritional supplements, drugs, chemicals and probiotics to easily test for agents that strengthen or destabilize the gut barrier.

Determine the Effect of Gut Microbes on Stress Polarity Pathway.

Rationale: Prior to this disclosure's work, the stress-polarity pathway had been characterized exclusively in MDCK monolayers [17, 44, 45]. To discern the importance of this pathway in the gut, the stress-polarity pathway in the colonic epithelial cell line Caco-2 can be interrogated; these cells form polarized monolayers in cultures with TJs that resemble those in the gut both functionally and morphologically [68]. Furthermore, butyrate and short-chain fatty acids stabilize TJs of Caco-2 monolayers via activation of AMPK [22, 23, 69].

General approach: Polarized Caco-2 monolayers (either WT, or lines stably depleted of GIV or AMPK; see Table 1; column 1) can be exposed to a wide variety of stressors (see Table 1; column 2) and can be assessed for TJ integrity (see Table 1; column 4) and markers of the stress-polarity pathway (pS245GIV and pAMPK by confocal IF). GIV and AMPK can be manipulated by downregulating GIV using spinoculation [70] with a lentiviral shRNA construct [71] and AMPKα[1 and 2] using CRISPR/Cas9. These experiments determine whether a wide variety of stressors activate the AMPK→GIV axis and whether their ability to disrupt TJs is accentuated when the AMPK→GIV axis is compromised. To pin-point the role of the AMPK→GIV axis, Caco-2 lines stably depleted of endogenous GIV and expressing GIV mutants that mimic constitutive phosphorylation (S245D) or non-phosphorylatable (S245A) states can be tested, as was done previously in MDCK lines [17]. Experiments in WT Caco-2 monolayers can also be repeated after modulating the stress-polarity pathway by pre-treatment with known direct or indirect activators of AMPK (see Table 1; column 3). The rationale for using multiple modulators of this pathway lies in the fact that at steady-state, gut barrier homeostasis is achieved by a fine antagonistic balance between TJ-protectors and TJ disruptors. It is understood that AMPK activators (such as but not limited to Metformin and AICAR), probiotics like A. muciniphila [18, 21, 72, 73] and Lactobacilli mixture (VSL #3) [74-77], and various nutritional supplements [22-25] (see Table 1; column 3) activate the stress-polarity pathway and serve as TJ protectors. Their ability to render protection can be tested in AMPK or GIV-depleted monolayers. These experiments reveal that some TJ protectors utilize the AMPK→GIV axis, while others do not. There can be multiple stressors (Table 1; column 2), and many ways to modulate the AMPK→GIV pathway (see Table 1; column 3); pathogenic E. coli and LPS as stressors, and Metformin, A. muciniphila, butyrate and L-glutamine as enhancers of the stress-polarity pathway can be prioritized.

Detailed Methods: Caco-2 cells are seeded at a concentration of 5×10⁵ on the upper side of polystyrene TRANSWELL inserts (3-μm pore size, 12-mm filters; Corning) in 500 μl of complete growth medium which contains minimum essential medium (MEM; Gibco) supplemented with 2 mM glutamine, 1 mM sodium pyruvate, 1× nonessential amino acids, penicillin-streptomycin (100 U/ml), and 10% fetal bovine serum; for 14 days for complete differentiation. The integrity of the cell monolayer are evaluated by measuring the transepithelial resistance (TEER) [78] before and after each treatment with a voltohmeter (See Table 1 for details of sources and concentrations of each reagent that was used). For all treatments to modulate the stress-polarity pathway, 16-18 h duration is optimal (FIGS. 4-5). The duration of exposure for each stressor is determined by serial TEER measurements. Adherent invasive E. coli are used as a model of pathogenic bacteria as they are associated with Crohn's disease [79]. An optimal condition is used where pathogenic as well as non-pathogenic bacteria are grown in Luria broth (LB) under aerobic conditions followed by oxygen-limiting conditions to keep their invasiveness [80, 81]. A. muciniphila (from ATCC) is grown as done previously [72]. Statistical analyses: All data are analyzed using Prism 5 (GraphPad Software, La Jolla, Calif., USA). Means are compared with Student's t-test or analysis of variance (Anova). p <0.05 is considered as statistically significant.

TABLE 1
Table of Model systems, Tools and Techniques for Studying the Stress-Polarity Pathway in Colon

| Model system/genetic | | Methods to modulate the | Methods to interrogate the |
|---|---|---|---|
| manipulations | Stressors | ′Stress Polarity Pathway′ | integrity of TJs |
| Cultured colonic Caco-2 cell | Invasive and | Metformin (Sigma); 1 μM | TJ Function: |
| line: | non-invasive | AICAR (Sigma), 2 mM | 1. Measurement of |
| 1. Wild-type (WT) | E. coli (strains | Compound C (EMD | Transepithelial electrical |
| 2. AMPKα1/α2-depleted by | AIEC LF82 (Gift | Millipore); 10-20 μM | resistance (TEER) using the |
| Crispr/Cas9 (Gift from Benoit | from Darfeuille- | Probiotics: | voltohmeter (World Precision |
| Viollet, INSERM, France) | Michaud, | Akkermansia muciniphila | Instruments, Sarasota, FL). |
| 3. GIV-depleted using | France), E. coli | (ATCC) | 2. Paracellular transport by |
| Lentiviral shRNA [71] | K12 (moi 10)) | Lactobacillus (VSL#3; | FITC-dextran (10 kD; Sigma), |
| 4. GIV-depleted cells | LPS from | Sigma-Tau) | TJ Structure: |
| expressing shRNA resistant | 026:B6 (L3491 | Nutritional Supplements: | 1. Confocal |
| GIV-WT and GIV mutant | Sigma); 100 | 1. Butyrate (Sigma); 5 mM | Immunofluorescence: |
| (S245A and S245D) constructs | ng/ml | [22] | Total and pS245-GIV, |
| Human colonic enteroid- | Hydrogen | 2. Polyunsaturated Fatty | phospho-AMPK, occludin, ZO- |
| derived monolayers: | Peroxide [H₂O₂) | Acids | 1. |
| 1. Wild Type (WT) | (H1009 Sigma); | (PUFA No.2; Sigma); 10 | 2. TEM for tight junction, and |
| 2. AMPK-depleted by shRNA | 100 μM | μM [82] | other features of loss of |
| 3. GIV-depleted by shRNA | | 3. L-glutamine (Sigma); 2 | polarity [VACs, brush-border, |
| | | mM [24] | etc.]. |

Define the role of the stress polarity pathway in the human gut.

Rationale: The findings can be extended to a model of cultured human enteroids (FIG. 4A) isolated from the biopsy specimens obtained during colonoscopy. Enteroid-derived monolayers (EDMs) can be generated from the cultured enteroids, and various stressors (see Table 1; column 2) can be used to test the integrity of TJs (see Table 1; column 4). EDMs are an ideal model for studying the gut barrier in vitro as they contain four different cell types, the epithelial, goblet, Paneth, and enteroendocrine cells, and mimic the gut barrier most closely among available model systems.

General Approach: As outlined above, EDMs (WT, AMPK-depleted, or GIV-depleted) are analyzed for TJ integrity (see Table 1; column 4) after exposure to stressors, with/without pre-treatment with enhancers of the stress-polarity pathway (see Table 1; column 3). Data (showcased in FIGS. 4-5) indicates that multiple stressors can indeed activate the AMPK→GIV axis. The experiments in AMPK/GIV-depleted EDMs demonstrate that AMPK and/or GIV are essential for stabilization of the gut barrier when exposed to these stressors. Once again, pathogenic E. coli, LPS, the probiotic A. muciniphila, and only those nutritional supplements that emerged from the Caco-2 screen above are prioritized as agents that require an intact AMPK→GIV signaling axis for their action on TJs.

Detailed Methods: Isolation of enteroids and generation of enteroid-derived monolayers (EDM): Crypts are isolated from human colonic biopsies by digesting tissue with Collagenase type I (2 mg/ml; Invitrogen), filtered with a cell strainer and washed with medium (DMEM/F12 with HEPES, 10% FBS), as outlined before [83]. The epithelial units are suspended in MATRIGEL (BD basement membrane matrix). Cell-MATRIGEL suspension (15 μl) are placed at the center of the 24-well plate on ice and placed on the incubator upside-down for polymerization (as shown in FIG. 4A). After 10 min, 500 μl of 50% conditioned media (prepared from L-WRN cells with Wnt3a, R-spondin and Noggin) containing 10 μM Y27632 (ROCK inhibitor) and 10 μM SB431542 (an inhibitor for TGF-β type I receptor) is added to the suspension. The medium is changed every 2 days and the enteroids are expanded and frozen in liquid nitrogen. The formation of spheroids is shown in FIG. 4A. To prepare EDMs, single cells from enteroids in 5% conditioned media are added to diluted MATRIGEL (1:30) as done before [84]. The EDMs are differentiated for 2 days in advanced DMEM/F12 media without Wnt3a but with R-spondin, Noggin, B27 and N2 supplements and ROCK inhibitor [85]. As expected, this results in a marked reduction in the expression of the stemness marker Lgr5 in EDMs [85].

Rationale: A phase 2b clinical trial using a delayed release formulation of Metformin (Metformin DR, which is designed to target the lower bowel and limit absorption into the blood) showed that Metformin works largely in the colon to lower blood glucose [55]. Metformin treatment directly impacts the colonic mucosa and the gut microbiome [18], which stabilizes epithelial TJs. Markers of the stress-polarity pathway (pAMPK and pS245GIV) can be assessed by IHC on FFPE (Formalin-Fixed Paraffin-Embedded) colonic biopsies from a retrospective cohort of age/sex matched veterans with insulin resistance/type II diabetes who were (or not; control) on Metformin alone as anti-diabetic regimen for >6 week duration. For 2-3 samples in which pAMPK/pGIV is absent or detected strongly, EDMs derived from those biopsies can be assessed for TJ integrity in vitro as outlined in the Table 1 (column #4). A pilot secondary analysis among patients taking Metformin can include correlation of the intensity of staining for pS245GIV with their glycemic control (Hemoglobin A1C; HbA1C). Other confounding factors (concurrent medications, illnesses) can be taken into account during data analysis. Findings can help design a clinical trial to determine if activation of the stress-polarity pathway by Metformin is a marker that distinguishes Metformin-responders from non-responders [86]. Because Metformin DR (Metformin delayed-release) is a drug uniquely suited for patients who cannot currently use Metformin owing to contraindications or poor tolerability (like renal failure, etc.), larger trials built on this foundation can allow more patients to access the beneficial actions of Metformin by simply taking the non-absorbable formulation.

Success benchmarks and interpretation of results. It is understood that Metformin, AICAR, and the commensals like A. muciniphila and the lactobacilli mixture VSL #3 protect TJ integrity (and therefore, the gut barrier) when challenged with stressors. Such protection can require an intact AMPK→GIV signaling axis, i.e., no protection can be seen in cells/EDMs depleted of either AMPKα[1 and 2] or GIV. Using phosphomimic/non-phosphorylatable GIV mutants (S245D/A) that have been extensively characterized previously [17], the role of phosphorylation of GIV by AMPK in such protection can be pinpointed. The molecular mechanisms downstream of phospho-GIV that stabilize TJs during stress that are defined using MDCK monolayers (see Schematic FIG. 2C) may not be investigated because the presence of pS245-GIV at TJs correlates tightly and consistently with high structural and functional TJ integrity so far, and hence pS245GIV is used as a surrogate ‘readout’ for an intact AMPK→GIV axis of signaling. In some instances (some stressors), discordancy can be seen where pS245GIV is indeed at the TJs, but TJ integrity is compromised. In those situations, AMPK-depleted and GIV-depleted lines and various pharmacologic AMPK modulators to can be used dissect if alternative pathways were in play to resist junctional collapse in those contexts.

As for Caco-2, these cells are chosen because they are easy to transfect; other alternatives include polarized monolayers of another colonic epithelial cell line, HT29 clone 19A. It is commonly understood that M cells are the major pathway for entrance of pathogens in the intestine. To understand the importance of M cells using Caco-2 cells, after 14 days of differentiation, Raji B lymphocytes can be added to the basolateral chamber underlying Caco-2 monolayers as done previously [68, 87]. Successful establishment of M-like cells can be confirmed by transmission electron microscopy [88]. The M-cells can be generated from the enteroids using RANKL as done by the Clevers group [89]. Lentiviral-transduction of the enteroids (for GIV depletion) using the spinoculation method used previously by the Clevers group [70] can also be successfully employed.

As for alternatives to the probiotic A. muciniphila, another widely-used formulation, the lactobacilli mixture, VSL #3 (from Sigma-Tau) can be used. VSL #3 protects the epithelial barrier and reinforces the gut barrier by increasing TJ proteins [90], mucin secretion [91], and by stimulating the production of β-defensin by IECs [92]. These and other mechanisms synergistically mediate the observed protective effect of VSL #3 in a variety of chronic disease states [75-77, 92-99]. Inflammation-mediated upregulation of AMPK as a cytosolic energy sensor [78] following exposure to stressors, including pathogenic infections, has not been ruled out.

The familiar IHC protocols [100-102] can be used. The antibodies can be highly specific and all be previously validated for use in immunocytochemistry. Patients having confounding medications or concurrent illnesses that cloud interpretation or make it impossible can be excluded from studies in accordance with this disclosure. This disclosure has accumulated a cohort of at least 20 patients and biopsy samples from patients who are on (n=10) or not on (n=10) Metformin therapy for 6 week or longer. This disclosure tested total and pS245-GIV antibodies and found the signals to be highly specific (data not shown).

This disclosure defines a fundamental homeostatic mechanism by which the gut barrier resists stress-induced collapse, and how Metformin or commensal microbes use that mechanism to protect the gut. This has led to novel strategies for tightening a leaky gut, which is a major driver of multiple allergic, autoimmune and metabolic diseases. The findings of the disclosure also impact approaches to various chronic diseases and has led to the development of technology for screening multiple drugs, chemicals, nutritional supplements and probiotics for their ability to disrupt or protect the gut barrier.

Define the Role of the Engulfment Pathway in the Human Gut.

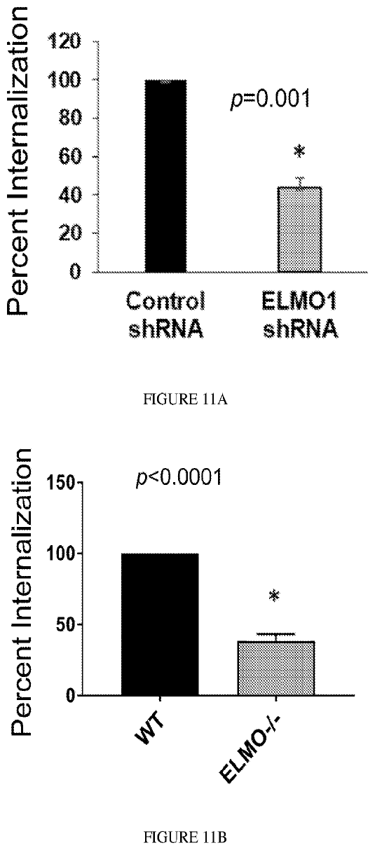

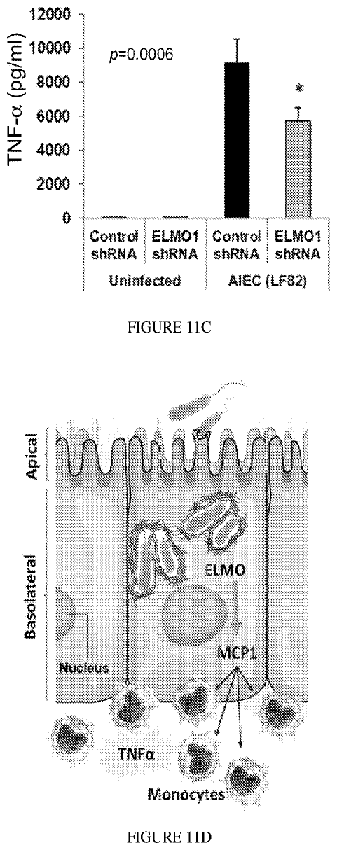

The present disclosure identifies the engulfment pathway that is coordinated by ELMO1 as a novel host response element, which operates in two different cell types in the gut, i.e., the epithelium and the monocytes. ELMO1 facilitates the engulfment of pathogenic microbes in the gut epithelium, and triggers the induction of the pro-inflammatory cytokine, MCP-1, the latter helping to recruit monocytes from peripheral blood to the site of local inflammation (see model FIG. 11D and FIGS. 10C and 10D). The disclosure highlights and exemplifies the complex multi-step interplay between luminal dysbiosis, which is an invariant hallmark in IBD and macrophages, and key players in the innate immune system of the gut. First, it was shown that the pathogenic AIEC-LF82 strain that is associated with CD can invade the gut epithelial lining by entering through epithelial TJs, and subsequently triggers the production of the pro-inflammatory cytokine, MCP-1 before being cleared via the phagolysosomal pathway. In the absence of ELMO1, uptake of the bacteria into the epithelial cells is impaired, and MCP-1 production is blunted. This ELMO1→MCP-1 axis then triggers the recruitment of monocytes. Next, it was shown that the same molecule, ELMO1 is also essential for the uptake and clearance of AIEC-LF82 in the monocytes, and is required for coordinately mounting yet another pro-inflammatory cytokine response, TNF-α. This ELMO1→TNF-α axis presumably feeds forward to propagate inflammation in the gut by triggering the activation of other monocytes and T-cells. Thus, the two signaling axes, ELMO1→MCP-1 and ELMO1→TNF-α, orchestrated by the same engulfment pathway in two different cell types, the epithelium and the monocytes, respectively, appear to be working as ‘first’ and ‘second’ responders to combat pathogenic microbes, thereby relaying distress signals from one cell type to another as the microbe invades through the breached mucosal barrier.

Until now, the majority of IBD-related research and therapeutic strategies have remained focused on T-cell responses and on neutralizing the impact of TNF-α. By demonstrating the presence of two hierarchical spatially and temporally separated signaling axes, the present disclosure provides mechanistic insights into some of the upstream/initial immune responses that play out in the epithelium and within the macrophages upon sensing luminal dysbiosis. This 3-way interaction between microbe-epithelium-macrophages is crucial to maintain homeostasis, and intestinal macrophages maintain the balance between homeostasis and inflammation [110]. A breach in the epithelium brought about by invading pathogens shifts the balance towards pro-inflammatory pathways. The present disclosure defines an upstream event that could be exploited to develop biomarkers, and eventually interrogated for the identification of strategies for therapeutic intervention (e.g., anti-MCP-1 therapy). The need for an in-depth understanding of the nature and the extent of the contribution of epithelial cells and/or monocytes in disease progression is urgent because of the limited efficacy of the available treatment options; for example, biologics that either neutralize TNF-α or prohibit the influx of T-cells to the gut lining are effective only in a third of the patients, and 40% of responders become refractory to treatment after 12 months [111]. Because the recruitment of monocytes from circulation to the site of infection/inflammation is a key early event in inflammatory diseases of the gut, the ELMO1→MCP-1 axis is potentially an actionable high value diagnostic and therapeutic target in IBD. Detection of high levels of ELMO1 in the epithelium could serve as an early indicator of activation of the engulfment pathway, and hence, could serve as a surrogate diagnostic marker of early inflammation due to luminal dysbiosis. Similarly, targeting the engulfment pathway is expected to restore immune homeostasis and resolve chronic inflammation via a completely novel approach that could synergize with existing therapies, and thereby, improve response rates and rates of sustained remission.

The present disclosure also provides the first mechanistic insights into how luminal dysbiosis initiates inflammation in the gut. Bacterial clearance and microbial dysbiosis are hallmarks of CD that control the outcome of innate immune responses. Healthy commensals like Bacteroidetes and Faecalibacterium prausnitzii are decreased in patients with CD, while pathogenic microbes like invasive Escherichia coli, Serratia marcescens, Cronobacter sakazakii and Ruminoccus gnavus are increased [112-115]. A dysbiotic microbial population can harbor pathogens and pathobionts that can aggravate intestinal inflammation or manifest systemic disease. Effector proteins produced by pathogenic bacteria can activate signaling that induce granuloma formation; one of the key symbols in CD pathogenesis [116]. In CD granuloma, the number of mucosal adherent invasive E. coli is higher because of defective clearance that can cause dysbiosis [117-118]. ELMO1 and the engulfment-pathway in the professional phagocytes have been shown to be essential for the internalization of Salmonella, and for mounting intestinal inflammation [100]. The present disclosure demonstrates the role of ELMO1 in non-phagocytic cells in the context of IBD using the CD-associated AIEC-LF82. The use of stem-cell based enteroids from ELMO1^{-/-} mice, either alone or in co-cultures with monocytes allowed for the interrogation of the function of ELMO1 in the epithelium and the monocytes separately.

Finally, by showing that the ELMO1→MCP-1 axis is an early step in gut inflammation, this work indicates the potential of anti-MCP-1 biologics to treat IBD. MCP-1 belongs to a CC chemokine subfamily, and its effects are mediated through CC chemokine receptor 2 (CCR2). So far, in human, only A2518G variation in MCP-1 gene promoter has been associated with CD [119]. However, MCP-1 is not just important in IBD, but also involved in other inflammatory diseases, such as atherosclerosis [120]. In fact, MCP-1 promotes the balance between anti-inflammatory and pro-inflammatory responses to infection. Treatment with recombinant MCP-1/CCL2 increases bacterial clearance and protects mice that are systemically infected with Pseudomonas aeruginosa or Salmonella typhimurium [121]. Administration of MCP-1 can increase chemotaxis on murine macrophages, enhance phagocytosis and killing of bacteria [121], whereas pretreatment of mice with anti-MCP-1/CCL2 impaired bacterial clearance. Therefore, increased expression of MCP-1 by ELMO1 in intestinal epithelium after exposure to AIEC-LF82 is likely to have a two-fold importance; (1) for controlling the increased bacterial load by killing the bacteria, and (2) for promoting monocyte recruitment and activation, which initiate a pro-inflammatory cytokine storm by inducing TNF-α from macrophages.
Expression of ELMO1 correlates positively with pro-inflammatory cytokines, MCP-1 and TNF-α.

ELMO1 has been shown to be involved in intestinal inflammation and microbial sensing [100].

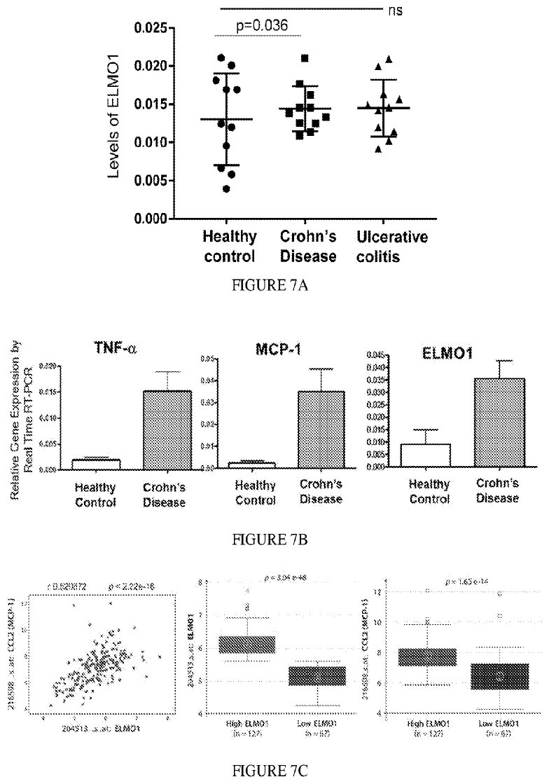

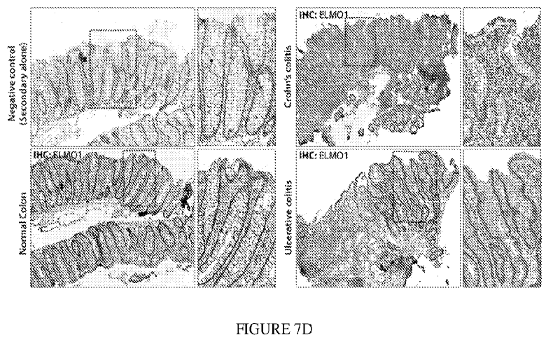

General Approach: To understand the role of ELMO1 in inflammatory bowel diseases, publicly available datasets (Pubmed; Gene Expression Omnibus (GEO) datasets GDS1330/24F24) were analyzed for expression of ELMO1 in the sigmoid colons of ulcerative colitis (UC) and Crohn's disease (CD) patients (FIG. 7A). In healthy humans, ELMO1 expression is heterogeneous with an average expression of ˜0.10 Arbitrary Units (AU). In patients with CD and UC however, expression is higher when compared to healthy humans (with p value of 0.036).

The relative expression of genes encoding pro-inflammatory cytokines TNF-α and MCP-1, namely CCL2 and ELMO1, were also assessed (FIG. 7B). RNA was isolated from human biopsy samples collected from colons of either healthy controls or those with active CD or CD in remission (n=6-8 samples/group). Compared to healthy controls, expression of both TNF-α and MCP-1 was elevated ˜6-fold in patients with active CD. As for ELMO1, its expression was elevated ˜4-fold compared to healthy controls.

General Approach: The association between the levels of ELMO1 and MCP-1 (CCL2) was studied using the publicly available NCBI-GEO data-series and analyzed using HEGEMON software (Hierarchical Exploration of Gene Expression Microarray Online) [122]. The publicly available RNA sequence data from 214 normal colons showed that ELMO1 and MCP-1 (CCL2) genes display a Boolean relationship in which, if the levels of expression of one is high, usually the other is also high (FIG. 7C). These findings suggest a more fundamental gene expression signature that is conserved despite population variance.

Detailed Approach: The association between the levels of ELMO1 and MCP-1 (CCL2) mRNA expression was tested in a cohort of normal colon tissue. This cohort included gene expression data from multiple publicly available NCBI-GEO data-series (10714, 10961, 11831, 12945, 13067, 13294, 13471, 14333, 15960, 17538, 18088, 18105, 20916, 2109, 2361, 26682, 26906, 29623, 31595, 37892, 4045, 4107, 41258, 4183, 5851, 8671, 9254, 9348), and contained information on 214 unique normal colon samples. All 214 samples contained in this subset were cross-checked to exclude the presence of redundancies/duplicates. To investigate the relationship between the mRNA expression levels of selected genes (i.e. ELMO1 and CCL2), the Hegemon software was applied. The Hegemon software is an upgrade of the BooleanNet software, where individual gene-expression arrays, after having been plotted on a two-axis chart based on the expression levels of any two given genes, can be stratified using the StepMiner algorithm and automatically compared for statistically significant differences in expression. The patient population of the NCBI-GEO discovery dataset were stratified in different gene-expression subgroups, based on the mRNA expression levels of ELMO1. Once grouped based on their gene-expression levels, patient subsets were compared for CCL2.

General Approach: To determine if elevated ELMO1 mRNA levels translate into elevated protein expression, and if so, which cell types contribute to such elevation immunohistochemistry (IHC) on colonic biopsies from healthy controls or patients with UC or CD was performed (FIG. 7D). ELMO1 was detected in both the epithelium and the lamina propria of the normal gut. In biopsies from patients with CD or UC, ELMO1 expression was elevated both in the epithelium and the lamina propria, but most strikingly in the diseased epithelium.

Detailed Approach: A total of 8 colonic specimens of known histologic type (3 normal colorectal tissue; 3 ulcerative colitis, and 2 Crohn's disease) were analyzed by IHC using anti-ELMO1 antibody (1:20, anti-rabbit antibody from Novus). Briefly, formalin-fixed, paraffin-embedded tissue sections of 4 μm thickness were cut and placed on glass slides coated with poly-L-lysine, followed by deparaffinization and hydration. Heat-induced epitope retrieval was performed using citrate buffer (pH 6) in a pressure cooker. Tissue sections were incubated with 0.3% hydrogen peroxidase for 15 min to block endogenous peroxidase activity, followed by incubation with primary antibodies for 30 min in a humidified chamber at room temperature. Immunostaining was visualized with a labeled streptavidin-biotin using 3,3′-diaminobenzidine as a chromogen and counterstained with hematoxylin. All the samples were first quantitatively analyzed and scored on the basis of 2 independent criteria. First, the intensity of staining was scored on a scale of 0 to 3, where 0=no staining, 1=light brown, 2=brown, and 3=dark brown. Second, the percentage of the cells that stained positive in the tumor area was scored on a scale of 0 to 4, where 0=0, 1=≤10%, 2=11-50%, 3=51-75%, and 4=>75%. Subsequently, each tumor sample was assigned a final score, which is the product of its (intensity of staining)×(% cells that stained positive). Tumors were categorized as negative when their final score was <3 and as positive when their final score was ≥3.

Taken together, these findings indicate that expression of ELMO1 is elevated in colons of patients with IBD.
The adherent-invasive E. coli (AIEC) as a model bacterium to study the role of the host engulfment pathway in CD pathogenesis.

Enteroid-derived monolayers (EDM) was used to investigate the role of ELMO1 in the IBD-afflicted gut epithelium. The use of human crypt-derived intestinal stem cells to develop enteroids for experimentation functionally recreates normal intestinal physiology.

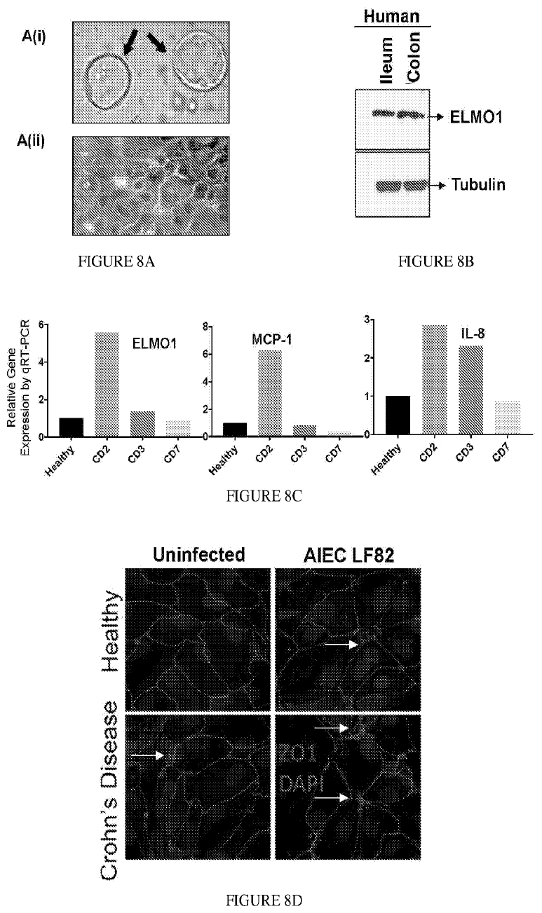

General Approach: Enteroids was generated (FIG. 8A i) from colonic biopsies obtained from healthy controls and CD patients, and enteroid-derived monolayers (FIG. 8A ii) (see Table 2 for patient demographics and clinical information).

TABLE 2

| | | Anatomic | | Treatment |
|---|---|---|---|---|
| Patient# | Age/Gender | location | Histopath | history |
| CD2 | 27/Female | Left | Colonic mucosa with | Remicade |
| | | colon | no diagnostic alteration. | 12 days |
| | | | No granulomas, viral | |
| | | | cytopathic effect or | |
| | | | dysplasia identified | |
| CD3 | 54/Male | Rectum | Colonic mucosa with no | Humira |
| | | | diagnostic alteration. | 6 days |
| CD7 | 19/Female | Left | Severely active colitis | Ustekinumab |
| | | colon | with ulceration, | At the time |
| | | | granulation tissue, and | of biopsy |
| | | | scattered granulomas | |

Detailed Approach: The colonic specimen was collected, washed in ice-cold PBS to remove fat and veins. Crypts were isolated from the tissue by digesting with Collagenase type I [2 mg/ml; Invitrogen], filtered with a cell strainer and washed with medium (DMEM/F12 with HEPES, 10% FBS). After adding collagenase I solution containing gentamicin (50 μg/ml, Life Technologies) and mixing thoroughly, the plate was incubated at 37° C. inside a CO₂ incubator for 10 min, with vigorous pipetting between incubations and monitoring the intestinal crypts dislodging from tissue. The collagenase was inactivated with media and filtered using a 70-μm cell strainer over a 50-ml centrifuge tube. Filtered tissue was spun down at 200 g for 5 min and the media was aspirated. The epithelial units were suspended in MATRIGEL (BD basement membrane matrix). Cell-MATRIGEL suspension (15 μl) was placed at the center of the 24-well plate on ice and placed on the incubator upside-down for polymerization. After 10 min, 500 μl of 50% conditioned media (prepared from L-WRN cells with Wnt3a, R-spondin and Noggin) containing 10 μM Y27632 (ROCK inhibitor) and 10 M SB431542 (an inhibitor for TGF-β type I receptor) were added to the suspension. For the human colonic specimens Nicotinamide (10 μM, Sigma-Aldrich), N-acetyl cysteine (1 mM, Sigma-Aldrich), and SB202190 (10 μM, Sigma-Aldrich) were added to the above media. The medium was changed every 2 days and the enteroids were expanded and frozen in liquid nitrogen.

To prepare EDMs, single cells from enteroids in 5% conditioned media was added to diluted MATRIGEL (1:30) as done before. The EDMs were differentiated for 2 days in advanced DMEM/F12 media without Wnt3a but with R-spondin, Noggin, B27 and N2 supplements and 10 μM ROCK inhibitor. As expected, this results in a marked reduction in the expression of the stemness marker Lgr5 in EDMs.

General Approach: The expression of ELMO1 in the enteroids was confirmed by immunoblotting (FIG. 8B) and by quantitative real-time RT-PCR (qRT-PCR; FIG. 8C). When compared to healthy controls, levels of ELMO1 mRNA were elevated in CD-derived enteroids (FIG. 8C i). Although the degree of elevation, was heterogeneous, as expected given that the patients were undergoing anti-TNF-α therapy just before biopsies were taken (see Table 2 column 5), the degree of increase in ELMO1 positively tracked with markers of inflammation, i.e., expression of MCP-1 and IL-8 (FIG. 8C ii-iii).

The role of epithelial ELMO1 in the generation of pro-inflammatory cytokine signature when exposed to luminal dysbiosis was investigated. To this end, adherent-invasive E. coli (AIEC-LF82 strain) was used as a model microbe because it is associated with pathogenesis of CD [123-124]. Because invasive microbes attack the integrity of epithelial tight junctions (TJs), trigger a redistribution of apical tight junction protein Zonula Occludens-1 (ZO-1) and thereby, breach the epithelial barrier function during invasion, it was investigated how epithelial TJs are altered when healthy or CD-derived enteroids are exposed to AIEC-LF82. TJs were clearly defined and intact in the uninfected healthy EDMs, but they were disrupted when EDMs were infected with AIEC-LF82 (FIG. 8D). In fact, the extent of disruption was almost similar (i.e., ˜90-95% area affected) to uninfected CD-derived EDMs at baseline. Upon infection with AIEC-LF82, the CD-derived EDM showed increased levels of ZO-1 at the TJs, which may be due to a short-term protective mechanism(s) that recruits ZO-1 to resist infection/stress-induced TJ collapse. These findings confirm that the CD-associated AIEC-LF82 strain can indeed disrupt epithelia TJs in the healthy epithelium, much like that seen in CD-derived EDMs at baseline.

Detailed Approach: Adherent Invasive Escherichia coli strain LF82 (AIEC-LF82), isolated from the specimens of Crohn's disease patient, was obtained from the lab of Arlette Darfeuille-Michaud. Non-invasive and non-pathogenic Escherichia coli K12 was used for infection where indicated as a negative control. For bacterial culture, a single colony was inoculated into LB broth and grown for 8 h under aerobic conditions in an orbital shaking incubator at 150 rpm and then under oxygen-limiting conditions overnight to keep their invasiveness. Cells were infected with a multiplicity of infection (moi) of 10.
ELMO1 is required for the engulfment of AIEC-LF82 within the gut epithelium.

Microbial dysbiosis is one of the major components in the pathogenesis of IBD [125-127]. Interactions of the invading microbe with the host cellular processes is a key trigger for the generation of inflammatory responses. Although phagocytic cells are primarily engaged in the uptake and clearance of microbes, it is well known microbes do enter through epithelial TJs. What is unknown is whether the epithelial cell relies on the engulfment pathway for uptake and subsequently clear them via the phagolysosomal pathway.

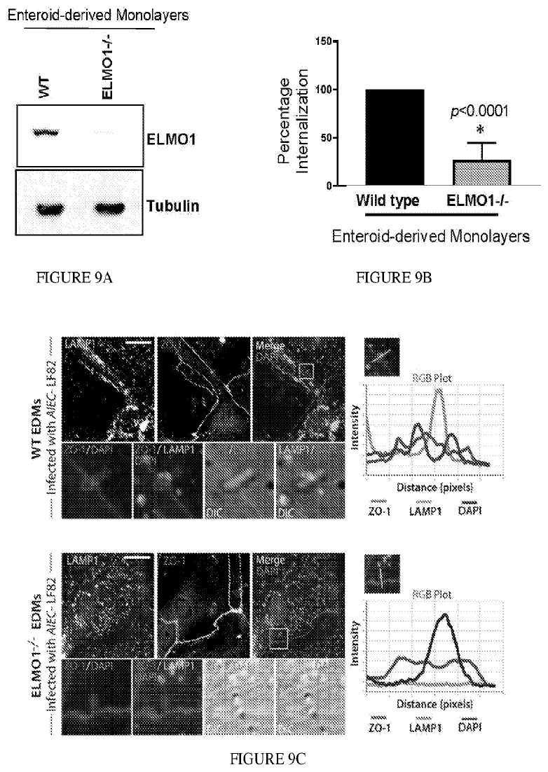

General Approach: To understand the role of ELMO1 during bacterial entry into epithelial cells, EDMs generated from colons of WT and ELMO1^{-/-} mice were used. Depletion of ELMO1 in the EDMs from ELMO1^{-/-} mice was confirmed by immunoblotting (FIG. 9A). When bacterial internalization was measured using the well-accepted gentamicin protection assay, it was found that compared to WT controls (n=10), internalization at 1 h after infection was decreased by 73% decrease in ELMO1 knock out EDMs (n=8) (p≤0.05; FIG. 9B).

Detailed Approach: Approximately 2×10⁵ cells were plated onto a 0.4 μm pore TRANSWELL insert and infected with bacteria with moi 10. Bacterial internalization was determined by gentamicin protection assay after infecting WT and ELMO1^{-/-} EDMs with AIEC-LF82 and treated with gentamicin after 1 h to remove extracellular bacteria. After 6 h of infection, cells were lysed cells with 1% Triton X-PBS, followed by serial dilutions with PBS and plated in LB agar tri-plate (VWR) and incubated overnight at 37° C. Bacterial colonies were counted after 16 h of incubation.

General Approach: Confocal immunofluorescence studies were also conducted to evaluate how the bacteria enter the epithelial cells. It was found that in both WT and ELMO1^{-/-} EDMs the AIEC-LF82 enter through the epithelial TJs, as determined by ZO-1 staining that surrounded the bacteria (FIG. 9C). However, dissimilarities of the proximity of lysosomes to the invading pathogens were found. In WT EDMs, lysosomes (as detected using the lysosomal integral membrane protein, LAMP1) were found in close proximity to the invading AIECs (FIG. 9C) in WT EDMs, indicating that lysosomes are recruited to the site of TJ breach. Whereas, such approximation was not seen in the ELMO1^{-/-} EDMs. These findings raise the possibility that in the absence of lysosome targeting, ELMO1^{-/-} EDMs may be defective not just in bacterial uptake, but also in bacterial clearance. These findings are consistent with a previously published role of ELMO1 in the clearance of another invasive pathogen, Salmonella [128].

Detailed Approach: WT and ELMO1^{-/-} EDMs were plated onto 8-well chamber slides (Millicell) and infected with bacteria with moi 10. After 1 h infection, media was aspirated, and cells were treated with 5% CM media with 250 μg/ml gentamicin for 90 min. Media was aspirated and 5% CM media was added to the wells. After 6 h of total infection time, samples were washed in 1-X PBS, pH 7.4 and fixed in 2% formaldehyde, washed with PBS, and permeabilized with 0.1% saponin-2% BSA (Sigma-Aldrich) in PBS for 10 minutes. Cells were blocked with 0.05% saponin-1% BSA in PBS (blocking solution) subsequently incubated with LAMP1 (Biolegend) and ZO-1 (Santa Cruz cat #sc-33725) overnight in blocking solution, diluted 1:800 and 1:140 respectively. The secondary antibodies, goat anti-mouse-Alexa488 (Life Technologies, cat #A-11017 1/500), goat anti-rabbit-Alexa594 (Life Technologies, cat #A-11012 1/500) and DAPI (1/1,000) were prepared in blocking solution. Images were acquired using a Leica TCS SPE CTR4000/DMI4000B-CS Confocal microscope with a Plan APO 63× objective. Multi-color images were obtained using excitation laser lines 405, 488 and 543 and transmission light, with respective detection. Z-stack acquisition was performed using a 1024×1024 pixels (58.3×58.3 micron) with a total of 10 sections (0.35 micron thickness). Images were analyzed in FIJI (FIJIJ is just ImageJ). Image flattening was obtained using average projection.

Taken together, the results using ELMO1^{-/-} EDMs demonstrate that ELMO1 is required for AIEC-LF82 uptake through breaches in the epithelial TJs, and for the proper targeting of lysosomes to the invading AIEC-LF82 pathogen. Morphologic findings in EDMs predict that once internalized, ELMO1 may also be required for efficient clearance of the AIEC-LF82 (studied in detail with macrophages in FIG. 11B).
ELMO1 in the gut epithelium is required for the generation of pro-inflammatory cytokine MCP-1.

It has been shown that ELMO1 and an intact host engulfment pathway is essential for the induction of pro-inflammatory cytokine MCP-1 in monocytes. Because the inflamed gut epithelium can express MCP-1, and because MCP-1 plays a major role in recruiting monocytes that in turn generates inflammatory cytokines in CD-afflicted gut, the role of ELMO1 for MCP-1 production by the gut epithelium once it is breached by invading AIEC-LF82 was investigated.

General Approach: The presence of any correlation between ELMO1 expression and MCP-1 in CD-derived EDMs was determined. To this end, the levels of MCP-1 by qRT-PCR (FIG. 10A) and by ELISA (FIG. 10B) were measured. In WT EDMs MCP-1 was undetectable without infection, but its levels were elevated after infection. Compared to WT EDMs, infection-triggered induction of MCP-1 was blunted in ELMO1^{-/-} EDMs (FIG. 10A-OB).

Detailed Approach: EDM layer following infection with AIEC-LF82 was collected for RNA isolation using RLT buffer (Qiagen Beverley, Inc.) and 0-mercaptoethanol. Total RNA was extracted using the RNeasy Microkit (Qiagen Beverly, Inc.) and reverse transcribed with a cDNA Supermix (Qiagen Beverly, Inc.), both according to the manufacturer's instructions and as done previously. Real-time RT PCR was performed using SYBR Green High ROX (Biotool) with primers (Integrated DNA Technologies, Inc.) detected using StepOnePlus Real-Time PCR Systems (Applied Biosystems) and normalized to the values of R-actin for mice and GAPDH for human. The fold change in mRNA expression was determined using the ΔΔCt method as done previously.

Supernatants were collected from the basolateral chamber either uninfected or after infected cells. MCP-1 was measured using the Mouse CCL2 (MCP-1) ELISA Ready-Set-Go Kit according to manufacturer's instructions (eBioscience). Supernatants were collected from control or ELMO1-depleted J774 cells after AIEC-LF82 infection and TNF-α was measured using the ELISA kit from BD bioscience.

Taken together, these findings demonstrate that ELMO1 is required for the generation of MCP-1 by the epithelium that is breached by CD-associated invasive pathogens, and that the ELMO1→MCP-1 axis may be a fundamental pathway that responds to dysbiosis in the gut lumen.
An intact ELMO1→MCP-1 axis in the gut epithelium is required for recruitment of monocytes.

Previous studies have demonstrated that CCL2/MCP-1^{-/-} mice had significant reduction in monocyte recruitment in inflammatory models and Th2 cytokines (IL-4, IL-5 and IFN-g) in the secondary pulmonary granulomata in response to Schistosoma mansoni eggs [129-130].

General Approach: To understand the role of the ELMO1-MCP-1 axis in the recruitment of monocytes, the WT and ELMO1^{-/-} EDMs were infected with AIEC-LF82 for 6 h and assessed the ability of these EDMs to recruit monocytes. After the infection, either the conditioned supernatant (FIG. 10C) or the infected monolayer itself (FIG. 10D) was co-cultured with monocytes, and migration of monocytes toward the infected EDM site was assessed. Compared to WT EDMs, ELMO1^{-/-} EDMs displayed a 50% reduction in monocyte recruitment. These results indicate that ELMO1-dependent MCP-1 production by the gut epithelium could serve as an upstream cue for monocyte recruitment to the sites of infection.

Detailed Approach: WT and ELMO1^{-/-} EDMs were plated in the TRANSWELL for polarization as mentioned previously, and infected with AIEC-LF82 for 6 h. Supernatant from the basolateral chamber was collected and placed in the new 24-well plate, and 6.5 mm 8-μm pore-sized TRANSWELLs (Costar) where THP-1 cells or peripheral blood-derived monocytes in OptiMEM (Gibco) were placed on the apical chamber. In another assay, the EDM layer was collected and flipped and placed on the bottom of the TRANSWELL. The number of recruited live monocytes were measured after 1, 2, 8, 16 and 24 h.

For some experiments, both 1 h and 6 h cells were collected for RNA isolation using RLT buffer (Qiagen Beverley, Inc.) and β-mercaptoethanol. Basolateral supernatant was collected for cytokine ELISAs. THP-1 (moi 20) cells were placed in the TRANSWELL, and live cells were collected and counted at 1 h, 2 h, 18 h, and 24 h time points. At 24 h post-addition of monocytes, basolateral supernatant was collected, pelleted down, and resuspended for total live monocyte cell counts.
ELMO1 in macrophages is essential for the engulfment of AIEC-LF82.

It was determined that ELMO1 impacts macrophage response upon being recruited to the sites of AIEC-LF82 infection. To study the role of ELMO1 in the internalization of AIEC-LF82, the gentamicin protection assay was used to assess bacterial uptake in ELMO1-depleted J774 macrophages (ELMO1 shRNA; around 90% depletion confirmed by immunoblotting). ELMO1-depleted cells showed approximately 50% reduction in bacterial internalization compared to WT cells (p value 0.001; FIG. 11A).

General Approach: To determine whether ELMO1 is essential for the clearance of AIEC-LF82 (as observed in Salmonella), bacterial engulfment and clearance was studied at 30 min, 3, 6, 12 and 24 h in the murine macrophage cell line J774. ELMO1^{-/-} cells showed lower uptake of AIEC-LF82 compared to WT macrophages (50% reduction; p value 0.001) at 30 min, but retention of a higher bacterial load at later time points (3 fold increase at 24 h after infection; with a p value 0.05). These findings indicate that ELMO1 is required not just for uptake, but also for clearance of AIEC-LF82.

To assess the contribution of ELMO1 in bacterial internalization in a more physiologically relevant system, the same assay was repeated, but the J774 cultured cell lines were replaced with primary intestinal macrophages enriched from WT or ELMO1^{-/-} mice (FIG. 11B). While intestinal macrophages from WT mice engulfed bacteria efficiently, bacterial uptake was decreased approximately 60% in macrophages from ELMO1^{-/-} mice (p value <0.0001), indicating that ELMO1 is essential for the engulfment of AIEC-LF82 in macrophages.

Because TNF-α is a major pro-inflammatory cytokine that is elevated early in the development of CD, the impact of reduced engulfment in the absence of ELMO1 on the release of TNF-α into the supernatant from control and ELMO1-depleted (by shRNA) J774 macrophages that were infected with AIEC-LF82 was analyzed. Using ELISA to detect the cytokine, it was found that, the ELMO1-depleted macrophages had significant reduction in TNF-α compared to control shRNA cells (p value 0.0006; FIG. 11C).

### Example Embodiments

In embodiments, this disclosure provides that Metformin (both absorbable Metformin and poorly absorbable Metformin (e.g. Metformin-DR; delayed release; marketed by Elcelyx)), analogues of the same, and/or other AMPK activators can be broadly used for multiple indications and for fixing a leaky gut barrier, which is a source of chronic endotoxemia and can fuel the progression of multiple chronic diseases, including but not limited to:
1) Metabolic syndrome
2) Obesity
3) Type II diabetes
4) Coronary artery disease
5) Fatty liver
6) Inflammatory bowel disease (Crohn's disease and ulcerative colitis)
7) Allergy (food allergy; celiac sprue)
8) Childhood allergy
9) Irritable bowel syndrome
10) Alzheimer's
11) Parkinson's
12) Autism
13) Colorectal cancer
14) Depression

In embodiments, the present disclosure provides methods effective to strengthen/protect the gut barrier and reduce and/or prevent the progression of chronic diseases. The gut barrier is a critical frontier that separates trillions of microbes and antigens from the largest immune system of the body; a compromised “leaky” gut barrier is frequently associated with systemic infection and inflammation, which is a key contributor to many chronic allergic, infectious and autoimmune diseases such as obesity, diabetes, inflammatory bowel diseases, food allergy, and metabolic endotoxemia.

In embodiments, tightening leaky gut is an effective way to inhibit systemic chronic endotoxemia, which drives many chronic diseases (e.g. allergic, autoimmune and infectious and metabolic, including obesity, fatty liver, type II DM, coronary artery disease, etc.). Metabolic diseases like type TT DM and obesity are diseases involving a leaky gut barrier, which can be reversed by giving a Metformin formulation that can work locally in the colon, not in systemic circulation.

In embodiments, the present disclosure provides methods for screening drugs, microbes, dietary components, nutritional supplements, substances of abuse (such as but not limited to nicotine, alcohol, e-cigarettes, cannabis), and pre- and probiotics for their ability to enhance or disrupt the gut barrier.

In embodiments, the present disclosure provides methods for screening drugs, microbes, toxins, dietary components, nutritional supplements, substances of abuse (such as but not limited to nicotine, alcohol, e-cigarettes, cannabis), and pre- and probiotics for their short-term inflammatory impact on the gut barrier.

In embodiments, the present disclosure provides methods for screening drugs, microbes, toxins, dietary components, nutritional supplements, substances of abuse (such as but not limited to nicotine, alcohol, e-cigarettes, cannabis), and pre- and probiotics for their long-term cancer sequelae.

In embodiments, the present disclosure provides methods and systems for screening to identify probiotics or compounds with beneficial effects on the gut barrier. In embodiments multi-well plates are used to create semi-high-throughput methods for screening drugs, microbes, toxins, dietary components, nutritional supplements, substances of abuse (such as but not limited to nicotine, alcohol, e-cigarettes, cannabis), and pre- and probiotics for their ability to enhance or disrupt the gut barrier. In embodiments multi-well plates are used to create semi-high-throughput methods for screening to identify probiotics or compounds with beneficial effects on the gut barrier (FIG. 17).

In embodiments, non-absorbable formulations of AMPK activators, such as Metformin-DR (Elcelyx), and probiotics like Akkermensia mucinalis are used for the treatment of diseases such as inflammatory bowel disease (ulcerative colitis and Crohn's disease), and metabolic syndrome spectrum (such as type II DM, obesity, and cardiovascular diseases).

In embodiments, the present disclosure determined a link between AMPK, use of AMPK agonists in disorders having impaired gut barrier at the center of their pathogenesis. In embodiments, the present disclosure provides for the use of Metformin and other AMPK agonists to treat disorders having impaired gut barrier.

In embodiments, the present disclosure determined a link between ELMO1, and the expression of MCP-1 and TNF-α in diseases having an inflammatory disorder at the center of their pathogenesis.

In embodiments, the present disclosure provides methods for screening drugs, nutritional supplements, and probiotics for their ability to enhance or disrupt the expression of MCP-1 in gut epithelium. In embodiments, the present disclosure provides methods and systems for screening to identify probiotics or compounds with beneficial effects on the expression levels of MCP-1.

In embodiments, the present disclosure provides methods for early detection of diseases associated with inflammation due to luminal dysbiosis.

### REFERENCES

1. Ohara, K, A Enomoto, T Kato, T Hashimoto, M Isotani-Sakakibara, N Asai, M Ishida-Takagishi, L Weng, M Nakayama, T Watanabe, K Kato, K Kaibuchi, Y Murakumo, et al. Involvement of Girdin in the determination of cell polarity during cell migration. PLoS One, 2012. 7(5): p. e36681.
2. Sasaki, K, T Kakuwa, K Akimoto, H Koga, and S Ohno. Regulation of epithelial cell polarity by PAR-3 depends on Girdin transcription and Girdin-Galphai3 signaling. J Cell Sci, 2015. 128(13): p. 2244-58.
3. Lee, J H, H Koh, M Kim, Y Kim, S Y Lee, R E Karess, S H Lee, M Shong, J M Kim, J Kim, and J Chung. Energy-dependent regulation of cell structure by AMP-activated protein kinase. Nature, 2007.447(7147): p. 1017-20.
4. Houssin, E, U Tepass, and P Laprise. Girdin-mediated interactions between cadherin and the actin cytoskeleton are required for epithelial morphogenesis in Drosophila. Development, 2015. 142(10): p. 1777-84.
5. Ghosh, P. The Stress Polarity Pathway: AMPK ‘GIV’-es protection against metabolic insults. Aging (Albany N.Y.), 2017; 9(2):303-314.
6. Molloy, M J, N Bouladoux, and Y Belkaid. Intestinal microbiota: shaping local and systemic immune responses. Semin Immunol, 2012. 24(1): p. 58-66.
7. Dominguez-Bello, M G, M J Blaser, R E Ley, and R Knight. Development of the human gastrointestinal microbiota and insights from high-throughput sequencing. Gastroenterology, 2011. 140(6): p. 1713-9.
8. Neish, A S. Microbes in gastrointestinal health and disease. Gastroenterology, 2009.136(1): p. 65-80.
9. Ley, R E, F Backhed, P Turnbaugh, C A Lozupone, R D Knight, and J I Gordon. Obesity alters gut microbial ecology. Proc Natl Acad Sci USA, 2005. 102(31): p. 11070-5.
10. Ley, R E, P J Turnbaugh, S Klein, and J I Gordon. Microbial ecology: human gut microbes associated with obesity. Nature, 2006. 444(7122): p. 1022-3.
11. Turnbaugh, P J, F Backhed, L Fulton, and J I Gordon. Diet-induced obesity is linked to marked but reversible alterations in the mouse distal gut microbiome. Cell Host Microbe, 2008. 3(4): p. 213-23.
12. Turnbaugh, P J and J I Gordon. The core gut microbiome, energy balance and obesity. J Physiol, 2009. 587(Pt 17): p. 4153-8.
13. Turnbaugh, P J, R E Ley, M A Mahowald, V Magrini, E R Mardis, and J I Gordon. An obesity-associated gut microbiome with increased capacity for energy harvest. Nature, 2006.444(7122): p. 1027-31.
14. Dumas, M E, R H Barton, A Toye, O Cloarec, C Blancher, A Rothwell, J Fearnside, R Tatoud, V Blanc, J C Lindon, S C Mitchell, E Holmes, M I McCarthy, et al. Metabolic profiling reveals a contribution of gut microbiota to fatty liver phenotype in insulin-resistant mice. Proc Natl Acad Sci USA, 2006. 103(33): p. 12511-6.
15. Tsukita, S, Y Yamazaki, T Katsuno, A Tamura, and S Tsukita. Tight junction-based epithelial microenvironment and cell proliferation. Oncogene, 2008. 27(55): p. 6930-8.
16. Cunningham, K E and J R Turner. Myosin light chain kinase: pulling the strings of epithelial tight junction function. Ann N Y Acad Sci, 2012. 1258: p. 34-42.
17. Aznar, N, A Patel, C C Rohena, Y Dunkel, L P Joosen, V Taupin, I Kufareva, M G Farquhar, and P Ghosh. AMP-activated protein kinase fortifies epithelial tight junctions during energetic stress via its effector GIV/Girdin. Elife, 2016. 5.
18. Shin, N R, J C Lee, H Y Lee, M S Kim, T W Whon, M S Lee, and J W Bae. An increase in the Akkermansia spp. population induced by metformin treatment improves glucose homeostasis in diet-induced obese mice. Gut, 2014. 63(5): p. 727-35.
19. Everard, A, C Belzer, L Geurts, J P Ouwerkerk, C Druart, L B Bindels, Y Guiot, M Derrien, G G Muccioli, N M Delzenne, W M de Vos, and P D Cani. Cross-talk between Akkermansia muciniphila and intestinal epithelium controls diet-induced obesity. Proc Natl Acad Sci USA, 2013. 110(22): p. 9066-71.
20. Lee, H and G Ko. Effect of metformin on metabolic improvement and gut microbiota. Appl Environ Microbiol, 2014. 80(19): p. 5935-43.
21. Li, J, S Lin, P M Vanhoutte, C W Woo, and A Xu. Akkermansia Muciniphila Protects Against Atherosclerosis by Preventing Metabolic Endotoxemia-Induced Inflammation in Apoe−/− Mice. Circulation, 2016. 133(24): p. 2434-46.
22. Miao, W, X Wu, K Wang, W Wang, Y Wang, Z Li, J Liu, L Li, and L Peng. Sodium Butyrate Promotes Reassembly of Tight Junctions in Caco-2 Monolayers Involving Inhibition of MLCK/MLC2 Pathway and Phosphorylation of PKCbeta2. Int J Mol Sci, 2016. 17(10).
23. Peng, L, Z R Li, R S Green, I R Holzman, and J Lin. Butyrate enhances the intestinal barrier by facilitating tight junction assembly via activation of AMP-activated protein kinase in Caco-2 cell monolayers. J Nutr, 2009. 139(9): p. 1619-25.
24. Wang, B, Z Wu, Y Ji, K Sun, Z Dai, and G Wu. L-Glutamine Enhances Tight Junction Integrity by Activating CaMK Kinase 2-AMP-Activated Protein Kinase Signaling in Intestinal Porcine Epithelial Cells. J Nutr, 2016. 146(3): p. 501-8.
25. Qiu, Y, M Wang, T Tan, X Yuan, and Y Ding. Effects of n-3 polyunsaturated fatty acids on hepatic tight junction after partial hepatectomy in rats. Transplant Proc, 2011.43(5): p. 1480-3.
26. Bischoff, S C, G Barbara, W Buurman, T Ockhuizen, J D Schulzke, M Serino, H Tilg, A Watson, and J M Wells. Intestinal permeability—a new target for disease prevention and therapy. BMC Gastroenterol, 2014. 14: p. 189.
27. Lee, S Y, S H Lee, E J Yang, E K Kim, J K Kim, D Y Shin, and M L Cho. Metformin Ameliorates Inflammatory Bowel Disease by Suppression of the STAT3 Signaling Pathway and Regulation of the between Th17/Treg Balance. PLoS One, 2015. 10(9): p. e0135858.
28. Xue, Y, H Zhang, X Sun, and M J Zhu. Metformin Improves Ileal Epithelial Barrier Function in Interleukin-10 Deficient Mice. PLoS One, 2016. 11(12): p. e0168670.
29. Barbara, G. Mucosal barrier defects in irritable bowel syndrome. Who left the door open? Am J Gastroenterol, 2006. 101(6): p. 1295-8.
30. Camilleri, M and H Gorman. Intestinal permeability and irritable bowel syndrome. Neurogastroenterol Motil, 2007. 19(7): p. 545-52.
31. Alam, M Z, Q Alam, M A Kamal, A M Abuzenadah, and A Haque. A possible link of gut microbiota alteration in type 2 diabetes and Alzheimer's disease pathogenicity: an update. CNS Neurol Disord Drug Targets, 2014. 13(3): p. 383-90.
32. Clairembault, T, L Leclair-Visonneau, E Coron, A Bourreille, S Le Dily, F Vavasseur, M F Heymann, M Neunlist, and P Derkinderen. Structural alterations of the intestinal epithelial barrier in Parkinson's disease. Acta Neuropathol Commun, 2015.3: p. 12.
33. Buscarinu, M C, B Cerasoli, V Annibali, C Policano, L Lionetto, M Capi, R Mechelli, S Romano, A Fornasiero, G Mattei, E Piras, D F Angelini, L Battisitni, et al. Altered intestinal permeability in patients with relapsing-remitting multiple sclerosis: A pilot study. Mult Scler, 2016.
34. Nouri, M, A Bredberg, B Westrom, and S Lavasani. Intestinal barrier dysfunction develops at the onset of experimental autoimmune encephalomyelitis, and can be induced by adoptive transfer of auto-reactive T cells. PLoS One, 2014. 9(9): p. e106335.
35. Yacyshyn, B, J Meddings, D Sadowski, and M B Bowen-Yacyshyn. Multiple sclerosis patients have peripheral blood CD45RO+ B cells and increased intestinal permeability. Dig Dis Sci, 1996. 41(12): p. 2493-8.
36. Samsam, M, R Ahangari, and S A Naser. Pathophysiology of autism spectrum disorders: revisiting gastrointestinal involvement and immune imbalance. World J Gastroenterol, 2014.20(29): p. 9942-51.
37. van De Sande, M M, V J van Buul, and F J Brouns. Autism and nutrition: the role of the gut-brain axis. Nutr Res Rev, 2014. 27(2): p. 199-214.
38. Sandek, A, I Bjarnason, H D Volk, R Crane, J B Meddings, J Niebauer, P R Kalra, S Buhner, R Herrmann, J Springer, W Doehner, S von Haehling, S D Anker, et al. Studies on bacterial endotoxin and intestinal absorption function in patients with chronic heart failure. Int J Cardiol, 2012. 157(1): p. 80-5.
39. Sandek, A, M Rauchhaus, S D Anker, and S von Haehling. The emerging role of the gut in chronic heart failure. Curr Opin Clin Nutr Metab Care, 2008. 11(5): p. 632-9.
40. Sandek, A, J Bauditz, A Swidsinski, S Buhner, J Weber-Eibel, S von Haehling, W Schroedl, T Karhausen, W Doehner, M Rauchhaus, P Poole-Wilson, H D Volk, H Lochs, et al. Altered intestinal function in patients with chronic heart failure. J Am Coll Cardiol, 2007. 50(16): p. 1561-9.
41. St Johnston, D and B Sanson. Epithelial polarity and morphogenesis. Curr Opin Cell Biol, 2011. 23(5): p. 540-6.
42. Wodarz, A and I Nathke. Cell polarity in development and cancer. Nat Cell Biol, 2007.9(9): p. 1016-24.
43. St Johnston, D and J Ahringer. Cell polarity in eggs and epithelia: parallels and diversity. Cell, 2010. 141(5): p. 757-74.
44. Zheng, B and L C Cantley. Regulation of epithelial tight junction assembly and disassembly by AMP-activated protein kinase. Proc Natl Acad Sci USA, 2007. 104(3): p. 819-22.
45. Zhang, L, J Li, L H Young, and M J Caplan. AMP-activated protein kinase regulates the assembly of epithelial tight junctions. Proc Natl Acad Sci USA, 2006. 103(46): p. 17272-7.
46. Cani, P D, M Osto, L Geurts, and A Everard. Involvement of gut microbiota in the development of low-grade inflammation and type 2 diabetes associated with obesity. Gut Microbes, 2012. 3(4): p. 279-88.
47. Cani, P D, S Possemiers, T Van de Wiele, Y Guiot, A Everard, O Rottier, L Geurts, D Naslain, A Neyrinck, D M Lambert, G G Muccioli, and N M Delzenne. Changes in gut microbiota control inflammation in obese mice through a mechanism involving GLP-2-driven improvement of gut permeability. Gut, 2009. 58(8): p. 1091-103.
48. Muccioli, G G, D Naslain, F Backhed, C S Reigstad, D M Lambert, N M Delzenne, and P D Cani. The endocannabinoid system links gut microbiota to adipogenesis. Mol Syst Biol, 2010. 6: p. 392.
49. Cani, P D, J Amar, M A Iglesias, M Poggi, C Knauf, D Bastelica, A M Neyrinck, F Fava, K M Tuohy, C Chabo, A Waget, E Delmee, B Cousin, et al. Metabolic endotoxemia initiates obesity and insulin resistance. Diabetes, 2007. 56(7): p. 1761-72.
50. Cani, P D, N M Delzenne, J Amar, and R Burcelin. Role of gut microflora in the development of obesity and insulin resistance following high-fat diet feeding. Pathol Biol (Paris), 2008. 56(5): p. 305-9.
51. Escobedo, G, E Lopez-Ortiz, and I Torres-Castro. Gut microbiota as a key player in triggering obesity, systemic inflammation and insulin resistance. Rev Invest Clin, 2014. 66(5): p. 450-9.
52. Gummesson, A, L M Carlsson, L H Storlien, F Backhed, P Lundin, L Lofgren, K Stenlof, Y Y Lam, B Fagerberg, and B Carlsson. Intestinal permeability is associated with visceral adiposity in healthy women. Obesity (Silver Spring), 2011. 19(11): p. 2280-2.
53. Spruss, A, G Kanuri, C Stahl, S C Bischoff, and I Bergheim. Metformin protects against the development of fructose-induced steatosis in mice: role of the intestinal barrier function. Lab Invest, 2012.92(7): p. 1020-32.
54. Zhou, Z Y, L W Ren, P Zhan, H Y Yang, D D Chai, and Z W Yu. Metformin exerts glucose-lowering action in high-fat fed mice via attenuating endotoxemia and enhancing insulin signaling. Acta Pharmacol Sin, 2016. 37(8): p. 1063-75.
55. Buse, J B, R A DeFronzo, J Rosenstock, T Kim, C Burns, S Skare, A Baron, and M Fineman. The Primary Glucose-Lowering Effect of Metformin Resides in the Gut, Not the Circulation: Results From Short-term Pharmacokinetic and 12-Week Dose-Ranging Studies. Diabetes Care, 2016.39(2): p. 198-205.
56. Wu, T, M Horowitz, and C K Rayner. New insights into the anti-diabetic actions of metformin: from the liver to the gut. Expert Rev Gastroenterol Hepatol, 2016: p. 1-10.
57. Bauer, P V and F A Duca. Targeting the gastrointestinal tract to treat type 2 diabetes. J Endocrinol, 2016. 230(3): p. R95-R113.
58. McCreight, L J, C J Bailey, and E R Pearson. Metformin and the gastrointestinal tract. Diabetologia, 2016. 59(3): p. 426-35.
59. Nahorski, M S, M Asai, E Wakeling, A Parker, N Asai, N Canham, S E Holder, Y C Chen, J Dyer, A F Brady, M Takahashi, and C G Woods. CCDCl88A mutations cause PEHO-like syndrome in humans and mouse. Brain, 2016. 139(Pt 4): p. 1036-44.
60. Asai, M, N Asai, A Murata, H Yokota, K Ohmori, S Mii, A Enomoto, Y Murakumo, and M Takahashi. Similar phenotypes of Girdin germ-line and conditional knockout mice indicate a crucial role for Girdin in the nestin lineage. Biochem Biophys Res Commun, 2012.426(4): p. 533-8.
61. Enomoto, A, N Asai, T Namba, Y Wang, T Kato, M Tanaka, H Tatsumi, S Taya, D Tsuboi, K Kuroda, N Kaneko, K Sawamoto, R Miyamoto, et al. Roles of disrupted-in-schizophrenia 1-interacting protein girdin in postnatal development of the dentate gyrus. Neuron, 2009. 63(6): p. 774-87.
62. Willenborg, C and R Prekeris. Apical protein transport and lumen morphogenesis in polarized epithelial cells. Biosci Rep, 2011.31(4): p. 245-56.
63. Low, S H, M Miura, P A Roche, A C Valdez, K E Mostov, and T Weimbs. Intracellular redirection of plasma membrane trafficking after loss of epithelial cell polarity. Mol Biol Cell, 2000. 11(9): p. 3045-60.
64. Vega-Salas, D E, P J Salas, and E Rodriguez-Boulan. Exocytosis of vacuolar apical compartment (VAC): a cell-cell contact controlled mechanism for the establishment of the apical plasma membrane domain in epithelial cells. J Cell Biol, 1988. 107(5): p. 1717-28.
65. Stelzner, M, M Helmrath, J C Dunn, S J Henning, C W Houchen, C Kuo, J Lynch, L Li, S T Magness, M G Martin, M H Wong, J Yu, and NIHISC Consortium. A nomenclature for intestinal in vitro cultures. Am J Physiol Gastrointest Liver Physiol, 2012. 302(12): p. G1359-63.
66. Davies, J M, R Santaolalla, and M T Abreu. Use of Cancer Stem Cells to Investigate the Pathogenesis of Colitis-associated Cancer. Inflamm Bowel Dis, 2016. 22(4): p. 976-83.
67. Foulke-Abel, J, J In, O Kovbasnjuk, N C Zachos, K Ettayebi, S E Blutt, J M Hyser, X L Zeng, S E Crawford, J R Broughman, M K Estes, and M Donowitz. Human enteroids as an ex-vivo model of host-pathogen interactions in the gastrointestinal tract. Exp Biol Med (Maywood), 2014. 239(9): p. 1124-34.
68. Kerneis, S, A Bogdanova, J P Kraehenbuhl, and E Pringault. Conversion by Peyer's patch lymphocytes of human enterocytes into M cells that transport bacteria. Science, 1997. 277(5328): p. 949-52.
69. Elamin, E E, A A Masclee, J Dekker, H J Pieters, and D M Jonkers. Short-chain fatty acids activate AMP-activated protein kinase and ameliorate ethanol-induced intestinal barrier dysfunction in Caco-2 cell monolayers. J Nutr, 2013. 143(12): p. 1872-81.
70. Koo, B K, D E Stange, T Sato, W Karthaus, H F Farin, M Huch, J H van Es, and H Clevers. Controlled gene expression in primary Lgr5 organoid cultures. Nat Methods, 2012. 9(1): p. 81-3.
71. Ghosh, P, N Aznar, L Swanson, I C Lo, I Lopez-Sanchez, J Ear, C Rohena, N Kalogriopoulos, L Joosen, Y Dunkel, N Sun, P Nguyen, and D Bhandari. Biochemical, Biophysical and Cellular Techniques to Study the Guanine Nucleotide Exchange Factor, GIV/Girdin. Curr Protoc Chem Biol, 2016. 8(4): p. 265-298.
72. Derrien, M, E E Vaughan, C M Plugge, and W M de Vos. Akkermansia muciniphila gen. nov., sp. nov., a human intestinal mucin-degrading bacterium. Int J Syst Evol Microbiol, 2004. 54(Pt 5): p. 1469-76.
73. Zhao, S, W Liu, J Wang, J Shi, Y Sun, W Wang, G Ning, R Liu, and J Hong. Akkermansia muciniphila improves metabolic profiles by reducing inflammation in chow diet-fed mice. J Mol Endocrinol, 2017. 58(1): p. 1-14.
74. Mennigen, R, K Nolte, E Rijcken, M Utech, B Loeffler, N Senninger, and M Bruewer. Probiotic mixture VSL #3 protects the epithelial barrier by maintaining tight junction protein expression and preventing apoptosis in a murine model of colitis. Am J Physiol Gastrointest Liver Physiol, 2009. 296(5): p. G1140-9.
75. Mencarelli, A, S Cipriani, B Renga, A Bruno, C D'Amore, E Distrutti, and S Fiorucci. VSL #3 resets insulin signaling and protects against NASH and atherosclerosis in a model of genetic dyslipidemia and intestinal inflammation. PLoS One, 2012. 7(9): p. e45425.
76. Kumar, M, V Kissoon-Singh, A L Coria, F Moreau, and K Chadee. Probiotic mixture VSL #3 reduces colonic inflammation and improves intestinal barrier function in Muc2 mucin-deficient mice. Am J Physiol Gastrointest Liver Physiol, 2017.312(1): p. G34-G45.
77. Chan, Y K, H El-Nezami, Y Chen, K Kinnunen, and P V Kirjavainen. Probiotic mixture VSL #3 reduce high fat diet induced vascular inflammation and atherosclerosis in ApoE(−/−) mice. AMB Express, 2016. 6(1): p. 61.
78. Scharl, M, G Paul, K E Barrett, and D F McCole. AMP-activated protein kinase mediates the interferon-gamma-induced decrease in intestinal epithelial barrier function. J Biol Chem, 2009. 284(41): p. 27952-63.
79. Darfeuille-Michaud, A, J Boudeau, P Bulois, C Neut, A L Glasser, N Barnich, M A Bringer, A Swidsinski, L Beaugerie, and J F Colombel. High prevalence of adherent-invasive Escherichia coli associated with ileal mucosa in Crohn's disease. Gastroenterology, 2004. 127(2): p. 412-21.
80. Das, S, K A Owen, K T Ly, D Park, S G Black, J M Wilson, C D Sifri, K S Ravichandran, P B Ernst, and J E Casanova. Brain angiogenesis inhibitor 1 (BAI) is a pattern recognition receptor that mediates macrophage binding and engulfment of Gram-negative bacteria. Proc Natl Acad Sci USA, 2011. 108(5): p. 2136-41.
81. Lee, M S and Y J Kim. Signaling pathways downstream of pattern-recognition receptors and their cross talk. Annu Rev Biochem, 2007. 76: p. 447-80.
82. Yan, X P, S Wang, Y Yang, and Y D Qiu. Effects of n-3 polyunsaturated fatty acids on rat livers after partial hepatectomy via LKB1-AMPK signaling pathway. Transplant Proc, 2011. 43(10): p. 3604-12.
83. Miyoshi, H and T S Stappenbeck. In vitro expansion and genetic modification of gastrointestinal stem cells in spheroid culture. Nat Protoc, 2013. 8(12): p. 2471-82.
84. den Hartog, G, R Chattopadhyay, A Ablack, E H Hall, L D Butcher, A Bhattacharyya, L Eckmann, P R Harris, S Das, P B Ernst, and S E Crowe. Regulation of Rac1 and Reactive Oxygen Species Production in Response to Infection of Gastrointestinal Epithelia. PLoS Pathog, 2016. 12(1): p. e1005382.
85. Sato, T, R G Vries, H J Snippert, M van de Wetering, N Barker, D E Stange, J H van Es, A Abo, P Kujala, P J Peters, and H Clevers. Single Lgr5 stem cells build crypt-villus structures in vitro without a mesenchymal niche. Nature, 2009. 459(7244): p. 262-5.
86. Mahrooz, A, H Parsanasab, M B Hashemi-Soteh, Z Kashi, A Bahar, A Alizadeh, and M Mozayeni. The role of clinical response to metformin in patients newly diagnosed with type 2 diabetes: a monotherapy study. Clin Exp Med, 2015. 15(2): p. 159-65.
87. Gullberg, E, M Leonard, J Karlsson, A M Hopkins, D Brayden, A W Baird, and P Artursson. Expression of specific markers and particle transport in a new human intestinal M-cell model. Biochem Biophys Res Commun, 2000. 279(3): p. 808-13.
88. des Rieux, A, V Fievez, I Theate, J Mast, V Preat, and Y J Schneider. An improved in vitro model of human intestinal follicle-associated epithelium to study nanoparticle transport by M cells. Eur J Pharm Sci, 2007.30(5): p. 380-91.
89. de Lau, W, P Kujala, K Schneeberger, S Middendorp, V S Li, N Barker, A Martens, F Hofhuis, R P DeKoter, P J Peters, E Nieuwenhuis, and H Clevers. Peyer's patch M cells derived from Lgr5(+) stem cells require SpiB and are induced by RankL in cultured “miniguts”. Mol Cell Biol, 2012. 32(18): p. 3639-47.
90. Dai, C, D H Zhao, and M Jiang. VSL #3 probiotics regulate the intestinal epithelial barrier in vivo and in vitro via the p38 and ERK signaling pathways. Int J Mol Med, 2012.29(2): p. 202-8.
91. Caballero-Franco, C, K Keller, C De Simone, and K Chadee. The VSL #3 probiotic formula induces mucin gene expression and secretion in colonic epithelial cells. Am J Physiol Gastrointest Liver Physiol, 2007. 292(1): p. G315-22.
92. Schlee, M, J Harder, B Koten, E F Stange, J Wehkamp, and K Fellermann. Probiotic lactobacilli and VSL #3 induce enterocyte beta-defensin 2. Clin Exp Immunol, 2008. 151(3): p. 528-35.
93. Chapman, T M, G L Plosker, and D P Figgitt. VSL #3 probiotic mixture: a review of its use in chronic inflammatory bowel diseases. Drugs, 2006. 66(10): p. 1371-87.
94. Chapman, T M, G L Plosker, and D P Figgitt. Spotlight on VSL #3 probiotic mixture in chronic inflammatory bowel diseases. BioDrugs, 2007. 21(1): p. 61-3.
95. Distrutti, E, J A O'Reilly, C McDonald, S Cipriani, B Renga, M A Lynch, and S Fiorucci. Modulation of intestinal microbiota by the probiotic VSL #3 resets brain gene expression and ameliorates the age-related deficit in LTP. PLoS One, 2014. 9(9): p. e106503.
96. Dubey, A P, K Rajeshwari, A Chakravarty, and G Famularo. Use of VSL [sharp]3 in the treatment of rotavirus diarrhea in children: preliminary results. J Clin Gastroenterol, 2008. 42 Suppl 3 Pt 1: p. S126-9.
97. Fedorak, R N, B G Feagan, N Hotte, D Leddin, L A Dieleman, D M Petrunia, R Enns, A Bitton, N Chiba, P Pare, A Rostom, J Marshall, W Depew, et al. The probiotic VSL #3 has anti-inflammatory effects and could reduce endoscopic recurrence after surgery for Crohn's disease. Clin Gastroenterol Hepatol, 2015. 13(5): p. 928-35 e2.
98. Miele, E, F Pascarella, E Giannetti, L Quaglietta, R N Baldassano, and A Staiano. Effect of a probiotic preparation (VSL #3) on induction and maintenance of remission in children with ulcerative colitis. Am J Gastroenterol, 2009. 104(2): p. 437-43.
99. Rincon, D, J Vaquero, A Hernando, E Galindo, C Ripoll, M Puerto, M Salcedo, R Frances, A Matilla, M V Catalina, G Clemente, J Such, and R Banares. Oral probiotic VSL #3 attenuates the circulatory disturbances of patients with cirrhosis and ascites. Liver Int, 2014. 34(10): p. 1504-12.
100. Das, S, A Sarkar, S S Choudhury, K A Owen, V Castillo, S Fox, L Eckmann, M R Elliott, J E Casanova, and P B Ernst. ELMO1 has an essential role in the internalization of Typhimurium into enteric macrophages that impacts disease outcome. Cell Mol Gastroenterol Hepatol, 2015. 1(3): p. 311-324.
101. Ghosh, P, J Tie, A Muranyi, S Singh, P Brunhoeber, K Leith, R Bowermaster, Z Liao, Y Zhu, B LaFleur, B Tran, J Desai, I Jones, et al. Girdin (GIV) Expression as a Prognostic Marker of Recurrence in Mismatch Repair-Proficient Stage II Colon Cancer. Clin Cancer Res, 2016.22(14): p. 3488-98.
102. Garcia-Marcos, M, B H Jung, J Ear, B Cabrera, J M Carethers, and P Ghosh.
Expression of GIV/Girdin, a metastasis-related protein, predicts patient survival in colon cancer. FASEB J, 2011.25(2): p. 590-9.
103. Das S, Sarkar A, Ryan K A, et al. Brain angiogenesis inhibitor 1 is expressed by gastric phagocytes during infection with Helicobacter pylori and mediates the recognition and engulfment of human apoptotic gastric epithelial cells. FASEB J 2014; 28:2214-24.
104. Fond A M, Lee C S, Schulman I G, et al. Apoptotic cells trigger a membrane-initiated pathway to increase ABCA1. J Clin Invest 2015; 125:2748-58.
105. Lee C S, Penberthy K K, Wheeler K M, et al. Boosting Apoptotic Cell Clearance by Colonic Epithelial Cells Attenuates Inflammation In Vivo. Immunity 2016; 44:807-20.
106. Parmar A S, Lappalainen M, Paavola-Sakki P, et al. Association of celiac disease genes with inflammatory bowel disease in Finnish and Swedish patients. Genes Immun 2012; 13:474-80.
107. Pezzolesi M G, Katavetin P, Kure M, et al. Confirmation of genetic associations at ELMO1 in the GoKinD collection supports its role as a susceptibility gene in diabetic nephropathy. Diabetes 2009; 58:2698-702.
108. Whitaker J W, Boyle D L, Bartok B, et al. Integrative omics analysis of rheumatoid arthritis identifies non-obvious therapeutic targets. PLoS One 2015; 10: e0124254.
109. Singh U P, Singh N P, Murphy E A, et al. Chemokine and cytokine levels in inflammatory bowel disease patients. Cytokine 2016; 77:44-9.
110. Mowat A M, Bain C C. Mucosal macrophages in intestinal homeostasis and inflammation. J Innate Immun 2011; 3:550-64.
111. Ahluwalia J P. Immunotherapy in inflammatory bowel disease. Med Clin North Am 2012; 96:525-44, x.
112. Forbes J D, Van Domselaar G, Bernstein C N. The Gut Microbiota in Immune-Mediated Inflammatory Diseases. Front Microbiol 2016; 7:1081.
113. Oberc A, Coombes B K. Convergence of External Crohn's Disease Risk Factors on Intestinal Bacteria. Front Immunol 2015; 6:558.
114. Chassaing B, Darfeuille-Michaud A. The commensal microbiota and enteropathogens in the pathogenesis of inflammatory bowel diseases. Gastroenterology2011; 140:1720-28.
115. Hager C L, Ghannoum M A. The mycobiome: Role in health and disease, and as a potential probiotic target in gastrointestinal disease. Dig Liver Dis 2017; 49:1171-1176.
116. Caprilli R, Frieri G. The dyspeptic macrophage 30 years later: an update in the pathogenesis of Crohn's disease. Dig Liver Dis 2009; 41:166-8.
117. Frank D N, St Amand A L, Feldman R A, et al. Molecular-phylogenetic characterization of microbial community imbalances in human inflammatory bowel diseases. Proc Natl Acad Sci USA 2007; 104:13780-5.
118. Baumgart M, Dogan B, Rishniw M, et al. Culture independent analysis of ileal mucosa reveals a selective increase in invasive Escherichia coli of novel phylogeny relative to depletion of Clostridiales in Crohn's disease involving the ileum. ISME J 2007; 1:403-18.
119. Palmieri O, Latiano A, Salvatori E, et al. The -A2518G polymorphism of monocyte chemoattractant protein-1 is associated with Crohn's disease. Am J Gastroenterol 2010; 105:1586-94.
120. Gerard C, Rollins B J. Chemokines and disease. Nat Immunol 2001; 2:108-15.
121. Nakano Y, Kasahara T, Mukaida N, et al. Protection against lethal bacterial infection in mice by monocyte-chemotactic and -activating factor. Infect Immun 1994; 62:377-83.
122. Dalerba P, Sahoo D, Paik S, et al. CDX2 as a Prognostic Biomarker in Stage II and Stage III Colon Cancer. N Engl J Med 2016; 374:211-22.
123. Martinez-Medina M, Aldeguer X, Lopez-Siles M, et al. Molecular diversity of Escherichia coli in the human gut: new ecological evidence supporting the role of adherent-invasive E. coli (AIEC) in Crohn's disease. Inflamm Bowel Dis 2009; 15:872-82.
124. Croxen M A, Law R J, Scholz R, et al. Recent advances in understanding enteric pathogenic Escherichia coli. Clin Microbiol Rev 2013; 26:822-80.
125. Manichanh C, Borruel N, Casellas F, et al. The gut microbiota in IBD. Nat Rev Gastroenterol Hepatol 2012; 9:599-608.
126. Miyoshi J, Chang E B. The gut microbiota and inflammatory bowel diseases. Transl Res 2017; 179:38-48.
127. Ni J, Wu G D, Albenberg L, et al. Gut microbiota and IBD: causation or correlation? Nat Rev Gastroenterol Hepatol 2017; 14:573-584.
128. Sarkar A, Tindle C, Pranadinata R F, et al. ELMO1 Regulates Autophagy Induction and Bacterial Clearance During Enteric Infection. J Infect Dis 2017; 216:1655-1666.
129. Lu B, Rutledge B J, Gu L, et al. Abnormalities in monocyte recruitment and cytokine expression in monocyte chemoattractant protein 1-deficient mice. J Exp Med 1998; 187:601-8.
130. Deshmane S L, Kremlev S, Amini S, et al. Monocyte chemoattractant protein-1 (MCP-1): an overview. J Interferon Cytokine Res 2009; 29:313-26.

What is claimed is:

1. A method of treating a disease associated with leaky gut barrier in a patient comprising:
administering to the patient a pharmaceutical composition comprising a therapeutically effective amount of an AMP-activated kinase (AMPK) agonist.

2. The method of claim 1, wherein the AMPK agonist is Metformin.

3. The method of claim 1, wherein the AMPK agonist is a Metformin analogue.

4. The method of claim 1, wherein the pharmaceutical composition is a delayed release formulation of Metformin.

5. The method of claim 1, wherein the disease is chronic endotoxemia.

6. The method of claim 1, wherein the disease is selected from metabolic syndrome, obesity, type II diabetes, coronary artery disease, fatty liver, an inflammatory bowel disease, Crohn's disease, ulcerative colitis, allergy, food allergy, celiac sprue, childhood allergy, irritable bowel syndrome, Alzheimer's disease, Parkinson's disease, colorectal cancer, depression, and autism.

7. The method of claim 1, wherein the disease is associated with systemic infection and inflammation from having a leaky gut barrier.

8. A method of identifying a compound with an ability to enhance or disrupt the gut barrier comprising:
combining a candidate compound with an enteroid-derived monolayer;
measuring or observing a signal associated with an AMPK→GIV stress-polarity pathway; and
determining that the candidate compound activated the AMPK→GIV stress-polarity pathway.

9. The method of claim 8, wherein the candidate compound is a synthetic or naturally occurring small molecule or protein, a nutritional supplement, a dietary component, a probiotic, a prebiotic, or a combination thereof.

10. The method of claim 8, wherein the candidate compound is a synthetic or naturally occurring toxin or a substance of abuse selected from the group comprising nicotine, alcohol, and cannabis.

11. The method of claim 8, wherein the enteroid-derived monolayer is human.

12. The method of claim 8, wherein the enteroid-derived monolayer comprises epithelial, goblet, Paneth, and enteroendocrine cells.

13. The method of claim 8, wherein tight junction function is measured or tight junctions are observed.

14. A method of screening a compound for an ability to enhance or disrupt the expression of MCP-1 in gut epithelium comprising:
combining a candidate compound with an enteroid-derived monolayer;
measuring or observing a signal associated with an ELMO1→MCP-1 signaling axis; and
determining whether the candidate compound activated the ELMO1→MCP-1 signaling axis.

15. The method of claim 14, wherein the candidate compound is a synthetic or naturally occurring small molecule or protein, a nutritional supplement, a dietary component, a probiotic, a prebiotic, or a combination thereof.

16. The method of claim 14, wherein the enteroid-derived monolayer is human.

17. The method of claim 14, wherein the enteroid-derived monolayer comprises epithelial, goblet, Paneth, and enteroendocrine cells.

18. A method of identifying a compound with an ability to enhance or disrupt the expression of TNF-α in macrophages in the gut comprising:
combining a candidate compound with an enteroid-derived monolayer;
measuring or observing a signal associated with an ELMO1→TNF-α signaling axis; and
determining that the candidate compound activated the ELMO1→TNF-α signaling axis.

19. The method of claim 18, wherein the candidate compound is a synthetic or naturally occurring small molecule or protein, a nutritional supplement, a dietary component, a probiotic, a prebiotic, or a combination thereof.

20. The method of claim 18, wherein the enteroid-derived monolayer is human.

21. The method of claim 18, wherein the enteroid-derived monolayer comprises epithelial, goblet, Paneth, and enteroendocrine cells.

22. A method of detecting a disease associated with inflammation due to luminal dysbiosis comprising:
obtaining an epithelium sample from a subject; and
detecting ELMO1 levels in the epithelium sample from the subject, wherein increased ELMO1 levels in the subject compared to a healthy control indicate the presence of a disease associated with inflammation due to luminal dysbiosis.

23. A method of treating a disease associated with luminal dysbiosis in a patient comprising:
administering to the patient a pharmaceutical composition comprising a therapeutically effective amount of a MCP-1 inhibiting agent.

24. The method of claim 23, wherein the disease is selected from an inflammatory bowel disease, Crohn's disease, and ulcerative colitis.

25. The method of claim 23, wherein the MCP-1 inhibiting agent is an anti-MCP-1 antibody.
